(19) United States
(12) Patent Application Publication
(10) Pub. No.: US 2024/0285727 A1
(43) Pub. Date: Aug. 29, 2024

(54) NOVEL THERAPEUTIC PEPTIDES FOR NEURODEGENERATION

(71) Applicant: UNIVERSITY OF SOUTHERN CALIFORNIA, Los Angeles, CA (US)

(72) Inventors: Pinchas Cohen, Los Angeles, CA (US); Brendan Miller, Los Angeles, CA (US); Su-Jeong Kim, Los Angeles, CA (US)

(73) Assignee: UNIVERSITY OF SOUTHERN CALIFORNIA, Los Angeles, CA (US)

(21) Appl. No.: 18/565,306

(22) PCT Filed: Jun. 3, 2022
(86) PCT No.: PCT/US2022/032223

§ 371 Date: Nov. 29, 2023

Related U.S. Application Data
(60) Provisional application No. 63/196,480, filed on Jun. 3, 2021.

(51) Int. Cl. A61K 38/17; A61K 38/10; A61P 25/28; C12Q 1/6883; G01N 33/68

(52) U.S. Cl. CPC A61K 38/1709; A61K 38/10; A61P 25/28; C12Q 1/6883; G01N 33/6896; C12Q 2600/156; G01N 2800/2821; G01N 2800/50

(57) ABSTRACT

Described herein is a novel, mitochondrial encoded, open reading frame that leads to the production of a new mitochondrial peptide called SHMOOSE. SHMOOSE is a 58-amino-acid peptide and, within its open reading frame, contains a genome-wide significant small nucleotide polymorphism (SNP) that markedly increased risk for Alzheimer's disease, brain structure, brain gene expression, and cognition. SHMOOSE increased neuronal-type cell survival and protected against amyloid beta toxicity. Metabolomic studies revealed a role for the peptide in energy optimization, whose dysfunction and dysregulation leads to cell death in physiologically notable regions of the brain in neurodegenerative diseases such as Alzheimer's and Parkinson's. Methods and compositions, including peptide analogues and derivatives, are described for treatment and diagnostics.

### CROSS-REFERENCE TO RELATED APPLICATIONS

This application includes a claim of priority under 35 U.S.C. § 119(e) to U.S. provisional patent application No. 63/196,480, filed Jun. 3, 2021, the entirety of which is hereby incorporated by reference.

### STATEMENT REGARDING FEDERALLY-SPONSORED RESEARCH OR DEVELOPMENT

This invention was made with government support under Grant Nos. AG055369, AG062693, AG061834, and AG068405, awarded by National Institutes of Health. The government has certain rights in the invention.

### REFERENCE TO SEQUENCE LISTING

The Sequence Listing submitted Jun. 3, 2022 as a text file named “SequenceListing-065715-000102WO00_ST25” created on Jun. 3, 2022 and having a size of 39,992 bytes, is hereby incorporated by reference.

### FIELD OF THE INVENTION

Described herein are methods and compositions related to mitochondrial peptides for use in treating metabolic related disease and compositions, such as neurodegenerative disease.

### BACKGROUND

Recent omics have revealed novel functional genomic elements in neurobiology and Alzheimer's disease (AD), but two components have yet to be rigorously examined: microproteins and mitochondrial DNA variation. Microproteins are biologically active peptides encoded by small open reading frames (sORFs). These peptides have been missed for decades due to computational power and biochemical limitations. Yet today, high resolution genomics and proteomics have revealed thousands of uncharacterized microproteins.

Microproteins represent an enormous opportunity to understand neurobiology. Several mitochondrial-encoded microproteins have been studied for the past twenty years. One such microprotein is humanin, a 24 amino-acid peptide that was cloned out of the occipital lobe of an Alzheimer's disease (AD) patient. Since its discovery, humanin has been found to attenuate AD pathology in part through its trimeric receptor signaling and amyloid beta toxicity protection. Recently, it has been reported that cognitive age and circulating human levels associated with a single nucleotide polymorphism (SNP) within the humanin sORF 9, suggesting other mitochondrial SNPs might influence uncharacterized microproteins.

Mitochondrial-derived peptides (MDPs) are a class of peptides encoded by mtDNA small open reading frames (ORFs). The 16,569 bp mitochondrial genome encodes for 13 large proteins involved in oxidative phosphorylation: ATP6, ATP8, CO1, CO2, CO3, CYB, ND1, ND2, ND3, ND4L, ND4, ND5, and ND6; and re-annotating the mitochondrial genome to include sORFs between 9 and 40 amino acids revealed hundreds putative MDP sORFs. Mitochondria are key actors in generating energy and regulating cell death. Mitochondria communicate back to the cell via retrograde signals that are encoded in the nuclear genome, or are secondary products of mitochondrial metabolism.

More recently, mitochondrial-derived peptides that are encoded by the mitochondrial genome have been identified as important actors in these regulatory processes. Mitochondrial-derived retrograde signal peptides are believed to aid in the identification of genes and peptides with therapeutic and diagnostic to treat human diseases.

### SUMMARY OF INVENTION

The following embodiments and aspects thereof are described and illustrated in conjunction with compositions and methods which are meant to be exemplary and illustrative, not limiting in scope.

Disclosed herein are compositions including a mitochondrial peptide having an amino acid sequence MPPCLTTWLSQLLKDNSYPLVLGPKNFGATPNKSNNHAHYYNHPNPD FPNSPHPYHPR (SEQ ID NO: 93), or a fragment, an analog, or a derivative thereof. In various embodiments, the mitochondrial peptide can include the amino acid sequence MPPCLTTWLSQLLKDNSYPLVLGPKNFGATPNKSNNHAHYYNHPNPDFPNSPHPYH PR (SEQ ID NO: 93). In various embodiments, the mitochondrial peptide can include an amino acid sequence of any of one of SEQ ID NO:1-SEQ ID NO:92, or SEQ ID NO:97-SEQ ID NO:107. In various embodiments, the mitochondrial peptide can include an amino acid sequence of PCLTTWLSQLLKDNSYPLVLGPKNF (SEQ ID NO: 3). In various embodiments, the mitochondrial peptide can include an amino acid sequence with about 20%, 25%, 30%, 35%, 40%, 45%, 50%, 55%, 60%, 65%, 70%, 75%, 80%, 85%, 90%, 95%, or more percentage identity to MPPCLTTWLSQLLKDNSYPLVLGPKNFGATPNKSNNHAHYYN HPNPDFPNSPHPYHPR (SEQ ID NO: 93), or to PCLTTWLSQLLKDNSYPLVLGPKNF (SEQ ID NO: 3). In various embodiments, the mitochondrial peptide can be 19-70 amino acids in length. In various embodiments, the mitochondrial peptide can possess a post-translational or artificial modification. For example, the artificial modification can include pegylation, fatty-acid conjugation, polypeptide extension, IgG-Fc, CPT, HSA, ELP, transferrin, or albumin modification. In various embodiments, the composition can further include a pharmaceutically acceptable excipient or pharmaceutically acceptable carrier.

Disclosed herein are methods of treating a disease and/or condition. In various embodiments, the method can include administering a quantity of a mitochondrial peptide to a subject in need of treatment of the disease and/or condition, wherein the mitochondrial peptide has an amino acid sequence of MPPCLTTWLSQLLKDNSYPLVLGPKNFGATPNKSNNHA HYYNHPNPDFPNSPHPYHPR (SEQ ID NO: 93), or a fragment, an analog, or a derivative thereof. In various embodiments, the mitochondrial peptide can be a fragment of the amino acid sequence of SEQ ID NO: 93. In various embodiments, the fragment can include the amino acid sequence PCLTTWLSQLLKDNSYPLVLGPKNF (SEQ ID NO: 3). In various embodiments, the mitochondrial peptide can be 19-70 amino acids in length. For example, the disease and/or condition can include a neurodegenerative disease and/or condition, optionally Alzheimer's disease or characterized by a level of amyloid beta above a reference. In various embodiments, the mitochondrial peptide increase tau levels in cerebrospinal fluid of the subject. In various embodiments, the mitochondrial peptide can decrease amyloid beta levels or amyloid beta plaques in a brain of the subject. In various embodiments, the mitochondrial peptide can reduce or inhibit a decrease in volume of interior temporal cortex tissue of the subject. In various embodiments, the subject can be a carrier of single nucleotide polymorphism (SNP) rs2853499 having an “A” allele at the SNP position. For example, the neurodegenerative disease and/or condition can be Parkinson's disease. In various embodiments, the mitochondrial peptide can reduce or inhibit decrease in volume of superior parietal lobe cortex tissue of the subject. In various embodiments, the subject can express high amounts of MPPCLTTWLSQLLKDNSYPLVLGPKNFGATPNKSNNHAHYYNHPNPDFPNSPHPYH PR (SEQ ID NO: 93) measured in a biological sample relative to a healthy normal subject.

Described herein are methods of detecting one or more biomarkers. In various embodiments, the method can include detecting the presence, absence, or expression level of one or more biomarkers in a biological sample obtained from a subject desiring a determination regarding the one or more biomarkers; and detecting the presence, absence, or expression level of the one or more biomarkers. In various embodiments, the one or more biomarkers can include a peptide of the sequence MPPCLTTWLSQLLKDNSYPLVLGPKNFGATPNKSNN HAHYYNHPNPDFPNSPHPYHPR (SEQ ID NO: 93), MPPCLTTWLSQLLKDNSYPLVLG PKNFGATPNKSNNHAHYYNHPNPNFPNSPHPYHPR (SEQ ID NO: 94), or a single nucleotide polymorphism (SNP) rs2853499. For example, detecting the presence, absence, or expression level includes an immunoassay. In various embodiments, the one or more biomarkers can include a single nucleotide polymorphism (SNP) rs2853499, wherein an “A” allele is at the SNP position. In various embodiments, the method can further include diagnosing the subject with a disease and/or condition or with an increased likelihood of having the disease and/or condition when the presence of the peptide having SEQ ID NO:94 is detected, OR diagnosing the subject with a disease and/or condition or with an increased likelihood of having the disease and/or condition when low expression levels of the peptide having SEQ ID NO:93, as compared to a healthy control, is detected, OR diagnosing the subject with a disease and/or condition when a single nucleotide polymorphism (SNP) rs2853499, wherein an “A” allele is at the SNP position, is detected. For example, the disease and/or condition is a neurodegenerative disease and/or condition. In various embodiments, the neurodegenerative disease and/or condition can be selected from the group consisting of Alzheimer's disease, Parkinson's disease, dementia, and combinations thereof.

Described herein are methods of detecting a genotype of a mitochondrial-derived peptide in a subject in need thereof. In various embodiments, the method can include assaying a biological sample obtained from the subject to detect genotype at a single nucleotide polymorphism (SNP). For example, the SNP is rs2853499. In various embodiments, the method can further include detecting an A allele at the SNP in the subject. For example, the subject has Alzheimer's disease or has a risk factor for developing Alzheimer's disease. In various embodiments, the detection can detect a count of the A allele at the SNP higher than that in a control subject, in the subject having Alzheimer's disease or has a risk factor for developing Alzheimer's disease. In various embodiments, the method can further include administering an Alzheimer's disease therapy to the subject. In various embodiments, the subject desires a determination regarding a neurodegeneration disease or disorder, optionally Alzheimer's disease.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

FIGS. 1A-1F depict that mitochondrial rs2853499 changes the amino acid sequence of SHMOOSE and associated with AD and neuroimaging modalities. Translational schematic showing how mitochondria DNA variants can be used to reveal a novel microprotein. (A) Visual representation of a mitochondrial single nucleotide polymorphism (“SNP”) that associates with Alzheimer's disease. SNP mutates the amino acids sequence of the peptide named “SHMOOSE,” resulting in “SHMOOSE SNP.” The GWAS Manhattan plot-equivalent of a MiWAS, called a Solar Plot. SNPs extending beyond the outer blue are statistically significant by a permutation empirical p value of 0.05. The most significant mtSNPs were rs2853498 and rs2853499—both are haplogroup U determining, with the latter causing the missense change to SHMOOSE. (B) Odds ratio of the SHMOOSE SNP in 4 cohorts (ADNI, ROSMAP, LOAD, ADC1/2). These cohorts are comprised of people with and without Alzheimer's disease (AD). Those people with the SHMOOSE SNP have a 30% greater chance of AD. Meta-analysis forest plot of rs2853499 in ADNI, ROSMAP, LOAD, and ADC/1/2. Bars represent 95% confidence intervals. (C) People with the same SHMOOSE SNP have faster atrophy of the hippocampus. This is highly significant to both AD and general neurodegeneration. Neuroimaging-based PheWAS in UK Biobank that illustrates the significant effects of SHMOOSE.D47N and age on parahippocampus. Other significant effects noted in for the EC and PCC in FIG. 12. (D) SHMOOSE SNP changes the 47th amino acid of the 58-amin-acid SHMOOSE peptide from Aspartic acid (D) to Asparagine (N). That is, rs2853499 changes the 47th amino acid of SHMOOSE. Computational-simulated models of SHMOOSE and SHMOOSE.D47N by RosettaTTFold. (E) When SHMOOSE expression is higher in the brain temporal cortex, so too are mitochondrial expression. GO cellular terms enriched by genes that co-express with SHMOOSE in the human temporal cortex (n=69). (F) SHMOOSE is detected in the nucleus and mitochondria of neuronal cells. Western blot detection of ˜6 kDa SHMOOSE in cells containing mtDNA versus cells not containing mtDNA (i.e., rho zero cells). Laminin B1 is a nuclear marker; GRSF1 isoform is a mitochondrial marker; GAPDH is a cytosolic marker.

FIGS. 2A-2C depict a new assay or test to quantify levels of SHMOOSE in biological tissues. An enzyme linked immunosorbent sandwich assay (ELISA) was developed by using an antibody against amino acids 32-58 of SHMOOSE. SHMOOSE levels in human cerebrospinal fluid (CSF) correlate to age, tau, and brain white matter microstructure. (A) RNA levels of SHMOOSE are higher in the temporal cortex of Alzheimer's disease brains. SHMOOSE RNA expression in the temporal cortex of AD cases. Significance represented as pAdj<0.05 following negative binomial regression on all normalized mitochondrial gene counts. (B) Actual peptide levels of SHMOOSE in cerebrospinal fluid correlate with age, tau, and phosphorylated tau. Tau and phosphorylated tau are Alzheimer's disease-related biomarkers. Human CSF SHMOOSE levels (pg/ml) correlation with age. Regression model includes biological sex as a covariate; p value<0.001. SHMOOSE correlation with CSF total tau (pg/ml). Regression model includes biological sex and age as covariates; p value<0.05. SHMOOSE correlation with CSF phosphorylated tau at residue 181 (p tau 181; pg/ml). Regression model includes biological sex and age as covariates; p value<0.05. (C) Actual peptide levels of SHMOOSE associates with worse brain white matter in the cingulate cortex, a region of the brain involved in many neurodegenerative diseases. Higher CSF SHMOOSE was significantly associated with lower DTI FA in the body of the corpus callosum and bilateral superior corona radiata in 72 non-demented older adults. Regression model included age, reported sex, and clinical dementia rating score. Colored voxels indicate FSLThreshold-Free Cluster Enhancement-derived p values<0.05 after correction for voxelwise multiple comparisons. Presented in radiological orientation (L=R).

FIGS. 3A-3C depict that SHMOOSE associates with differential mitochondrial and ribosomal gene expression in humans, cells, and mice. Solid-lined boxes represent mitochondrial terms and broken-lined boxes represent ribosomal terms. (A) depicts those humans with the SHMOOSE SNP (the same SNP that associates with Alzheimer's) also associates with differential mitochondrial and ribosomal gene expression. Principal component analysis (PCA), color coded for 14 SHMOOSE.D47N carriers or 55 SHMOOSE reference allele carriers. Dashed line represents the median value of PC2. Of the 14 SHMOOSE.D47N carriers, 11 fall below the median PC2 value (p value<0.05; generalized linear model). To the right of the PCA figure are GO cellular compartment terms enriched by SHMOOSE.D47N carriers. (B) depicts that the mutant form of SHMOOSE (SHMOOSE.D47N) causes changes in mitochondrial inner membrane gene expression for neuronal cells as well as ribosomal gene expression. PCA of the in vitro gene expression signature for 10 uM SHMOOSE or SHMOOSE.D47N-treated neural cells after 24 hours. To the right of the PCA figure are GO cellular compartment terms enriched by SHMOOSE.D47N-treated neural cells. (C) depicts that when SHMOOSE is injected into a mouse, ribosomal and mitochondrial gene expression is also changed. Corresponding PCA plots and GO cellular compartment terms, on liver tissue, enriched by SHMOOSE following 14-day SHMOOSE treatment to mice.

FIGS. 4A-4F depict the effect of SHMOOSE on mitochondrial energetics. SHMOOSE is a biologically active microprotein that localizes to mitochondria and boosts metabolic activity and oxidative consumption rate. (A) SHMOOSE, when given to cells, goes to the mitochondria. SHMOOSE-treated differentiated SH-SY5Y cells (1 uM) localized to mitochondria after 15 minutes. Top portion of the blot represents a 5-second exposure. Second portion of the blot represents a 30-second exposure. Also identified were SHMOOSE dimers around 12 kDa in the SHMOOSE-treated conditions that were most prominent in mitochondrial fractions. Laminin, GRSF1, and GAPDH represent nuclear, mitochondrial, and cytosolic fractions, respectively. (B) SHMOOSE, when given to cells, boosts metabolic activity in a dose response manner. There is no difference in this bulk metabolic activity between SHMOOSE and mutant SHMOOSE.D47N. Again, this is the version of SHMOOSE that associates with Alzheimer's. MTT assay results that illustrate 1 uM of both SHMOOSE (light blue) and SHMOOSE.D47N (dark blue) both have effects on cell metabolic activity at 1 uM and 10 uM. Significance defined as p value<0.05 for independent t tests. (C) SHMOOSE, but not mutant SHMOOSE.D47N, boosts basal cell oxygen consumption rate. Normalized to the baseline third measurement, the effect of SHMOOSE and SHMOOSE.D47N on mitochondrial spare capacity. Statistical significance determined using independent t tests. (D) SHMOOSE and mutant SHMOOSE.D47N boost the mitochondria's ability to handle stress. Effect of SHMOOSE and SHMOOSE.D47N on basal OCR. Significance defined as p value<0.05 for independent t tests. (E) SHMOOSE expression is highest in neurons with familial Alzheimer's mutations. SHMOOSE expression in neurons derived from iPSCs with FAD APP mutation, FAD PSEN mutation, and FAD APP plus PSEN mutations. SHMOOSE expression was highest in the latter cell type. Significance defined as pAdj<0.05 following negative binomial regression on all normalized mitochondrial gene counts. (F) SHMOOSE, but not mutant SHMOOSE.D47N, protects against amyloid beta in neuronal cells. SHMOOSE-treated neural cells protects against amyloid beta42-induced toxicity. Significance defined as p value<0.05 for independent t tests.

FIGS. 5A-5E depict various characteristics of SHMOOSE. SHMOOSE binds the inner mitochondrial membrane protein mitofilin. (A) SHMOOSE is part of binding complexes that consist of 98 proteins. One of these proteins is called mitofilin, which lives in the inner mitochondrial membrane. Schematic proteomics analysis of SHMOOSE-spiked neural cell lysates that were immunoprecipitated using a custom SHMOOSE polyclonal antibody. (B) SHMOOSE co-immunoprecipitates with mitofilin in neuronal cells. Reciprocal Western blot validation of SHMOOSE/mitofilin interaction by immunoprecipitating with SHMOOSE antibody or mitofilin antibody. (C) Synthetic SHMOOSE binds mitofilin outside of a cell. Reciprocal dot blot illustrating recombinant mitofilin and SHMOOSE interact. (D) When mitofilin levels are lowered in cells, the effects of SHMOOSE on metabolic activity goes down. MTT assay showing SHMOOSE has no effect of neural cell metabolic activity when mitofilin is knocked down with siRNA. Y axis is normalized to control baseline absorbance values. Statistical significance determined using independent t test with p value<0.05. (E) A computational simulation of SHMOOSE binding the N-terminus of mitofilin. HDOCK prediction of the SHMOOSE (yellow) and mitofilin (brown) interaction. Predicted interaction residues for mitofilin occur at its c-terminus.

FIG. 6A depicts that the liver transcriptome differentiates SHMOOSE-treated mice from control after 2 weeks, FIG. 6B depicts that SHMOOSE decreases liver enzymes AST and ALT in mice after 2 weeks, FIG. 6C depicts SHMOOSE attenuates weight gain on mice fed a high fat diet after 2 weeks, FIG. 6D depicts the hypothalamus transcriptome differentiates SHMOOSE-treated mice from control after 2 weeks, FIG. 6E depicts that the hypothalamus transcriptome after SHMOOSE treatment indicates potent ribosome and mitochondrial gene expression changes, and FIG. 6F depicts that SHMOOSE activates hypothalamic neurons as measured by cFOS.

FIG. 7 depicts the effect of SHMOOSE on mitochondrial superoxide. SHMOOSE reduces mitochondrial superoxide. IMMT siRNA reduces mitochondrial superoxide and SHMOOSE then has no affects. When mitofilin levels are lowered in cells, the effect of SHMOOSE on lowering reactive oxygen species is attenuated.

FIG. 8A depicts that SHMOOSE was detected in HEK293 cell mitochondria using a proximity mitochondria labeling strategy. Peptide sequence DNSYPLVLGPK (SEQ ID NO: 96) is shown in FIG. 8A. FIG. 8B depicts that SHMOOSE lives in the inner mitochondrial membrane.

FIG. 9A depicts that when SHMOOSE is given to neuronal cells, it goes to the mitochondria and binds mitochondrial supercomplexes, most prominently to ATP5. FIG. 9B depicts that when SHMOOSE is sucked out of a cell using immunoprecipitation, ATP5 is stuck to it. SHMOOSE binds ATP5. FIG. 9C depicts that SHMOOSE and the ATP5 subunits B and O bind outside of a cell. FIG. 9D is a visual representation of the proteins SHMOOOSE binds in ATP5, ATP50 and ATP5B binding, but ATP5A1 not binding.

FIG. 10 depicts an analogue screening assay, measured by MTT. Top: each bar represents a fragment (25-amino acid in length) of SHMOOSE. Bottom: each bar represents a phosphomimetic fragment (25-amino acid in length) of SHMOOSE. Cell model is SHSY5Y. The Inventors made 103 analogues/derivatives (see Table 1).

FIG. 11 depicts exemplary analogues of SHMOOSE with boosted activity. FIG. 11 shows an exemplary analogue of the SHMOOSE peptide having a sequence having 14 amino acids: PCLTTWLSQLLKDN (SEQ ID NO: 95). From the sequence of SEQ ID NO: 95, 3 different analysis windows of the analogue, PCLTTWLSQLL (SEQ ID NO: 108), CLTTWLSQLLK (SEQ ID NO: 109), and LTTWLSQLLKD (SEQ ID NO: 110), each having 11 amino acids, were characterized. Hydrophobic faces are formed by amino acids CWLLLL (SEQ ID NO: 111) from the sequences of SEQ ID NO: 108 and SEQ ID NO: 109. A hydrophobic face is formed by amino acids WLLLL (SEQ ID NO: 112) from the sequence of SEQ ID NO: 110.

FIG. 12 depicts Neuroimaging-based PheWAS in UK Biobank that illustrates the significant effects of SHMOOSE.D47N and age on respective neuroimaging markers. SHMOOSE.D47 significantly associated with cortical thickness, volume, pial surface area, WM surface Jacobian, and GM/WM contrast in several paralimbic regions, including the parahippocampal gyri, the entorhinal cortex (EC), the anterior cingulate cortex (ACC), the posterior cingulate cortex (PCC), and the temporal pole (TPO) (clusterwise, RFT-corrected p value<0.05. Color represents p value.

FIG. 13 depicts that SHMOOSE mtSNP associates with faster cognitive decline. Individuals with the SHMOOSE mtSNP (A allele) were predicted to have accelerated cognitive decline. Model shows effects estimated from a mixed effects growth model. Red trajectories represent SHMOOSE.D47N carriers. Effects estimated starting at age 65 years old.

FIG. 14 depicts a standard curve of SHMOOSE ELISA. The range of the standard curve is 100-250,000 pg/ml.

FIG. 15 depicts effects of p Tau 181 on SHMOOSE with tau as a mediator. The indirect effect (ACME) is not significant (−0.15) below the combined indirect and direct (ADE) (15.83; p value<0.01). The effect of p tau 181 on total was significant (6.025; p value<0.01), while the effect of total tau on SHMOOSE when controlling for p tau 181 was not significant (−2.56).

FIG. 16 depicts principal component analysis (PCA) color coded to represent the 8 APOE4 carriers. Dashed line represents the median value of PC2. While not statistically significant, 5 of the 8 APOE4 carriers fall below the PC2 median.

FIGS. 17A-17C depict PCA and GO Ontology Enrichment for cortex, hypothalamus, and hippocampus. (A) In cortex, PCA sufficiently separated SHMOOSE-treated mice from vehicle with cellular component terms enriched for CNS specifics. (B) In hypothalamus, PCA sufficiently separated SHMOOSE-treated mice from vehicle with cellular component terms enriched for CNS specifics. (C) In hippocampus, PCA did not sufficiently separate SHMOOSE-treated mice and did not enrich terms.

FIGS. 18A-18D depict effects of SHMOOSE on mice injected. (A) After two weeks, the AST levels of SHMOOSE-treated mice was lower (p value<0.1) compared to control treated mice on a high fat diet. (B) Likewise, ALT levels were lower in SHMOOSE-treated mice (p value<0.2). (C) SHMOOSE attenuated weight gain compared to control. (D) SHMOOSE did not change food intake.

FIGS. 19A-19D depict mitochondrial-DNA principal component analyses per cohort. (A) mtPCA within the ADNI cohort. SHMOOSE A allele carriers cluster together. (B) RUSH ROSMAP mtPCA. SHMOOSE A allele carriers also appear to cluster together. (C) mtPCA within the LOAD cohort. SHMOOSE A allele carriers are within three distinct clusters. (D) mtPCA within the ADC1/2 cohort. SHMOOSE A allele carriers are within three distinct clusters.

FIGS. 20A-20I depict the effects of SHMOOSE.D47N in the language centers (superior temporal and inferior frontal gyri), dorsolateral and medial prefrontal cortex, central motor, and occipital visual cortices in UKB. (A-I) In order, white matter surface area, pial surface area, surface jacobian, gray/white matter contrast, cortical thickness, cortical volume, sulcal depth, mean curvature, and gaussian curvature modeled. Grayscale indicates p value. Effects are shown with lenient, uncorrected p value<0.05.

FIGS. 21A-21I depict effects of SHMOOSE.D47N limbic regions such as the medial temporal cortex and posterior cingulate cortex at a lenient threshold of uncorrected p value<0.05 in ADNI. (A-I) In order, white matter surface area, pial surface area, surface jacobian, gray/white matter contrast, cortical thickness, cortical volume, sulcal depth, mean curvature, and Gaussian curvature modeled. Grayscale indicates p value. Effects are shown with lenient, uncorrected p value.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); and Sambrook and Russel, Molecular Cloning: A Laboratory Manual 4^{th} ed., Cold Spring Harbor Laboratory Press (Cold Spring Harbor, N Y 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“Administering” and/or “administer” as used herein refer to any route for delivering a pharmaceutical composition to a patient. Routes of delivery may include non-invasive peroral (through the mouth), topical (skin), transmucosal (nasal, buccal/sublingual, vaginal, ocular and rectal) and inhalation routes, as well as parenteral routes, and other methods known in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

“Analog” when used herein in reference to the SHMOOSE peptide of the present invention refers a peptide fragment of SHMOOSE. In various embodiments, the analogs have about the same or increased activity as compared to the reference peptide. In various embodiments, the increased activity is at least a 5% increase in activity. In various embodiments, the increased activity is at least 10%, 15%, 20%, 25%, 30%, 35%, 40%, 45%, 50%, 60%, 70%, 80%, 90%, 100%, 110%, 120%, 130%, 140%, 150%, 175%, or 200% increase in activity.

“Derivative” when used herein refers to a peptide that was designed based on the reference peptide. In various embodiments, the derivative peptide can have about the same or increased functional activity as the reference peptide. In various embodiments, the increased activity is at least 10%, 15%, 20%, 25%, 30%, 35%, 40%, 45%, 50%, 60%, 70%, 80%, 90%, 100%, 110%, 120%, 130%, 140%, 150%, 175%, or 200% increase in activity. In various embodiments, the derivative comprises one or more amino acid substitutions, deletions or additions. In various embodiments, the derivative peptide comprises up to 15 amino acid substitutions, deletions or additions. In various embodiments, the derivative peptide comprises are up to 10 amino acid substitutions, deletions or additions. In various embodiments, the derivative peptide comprises up to 5 amino acid substitutions, deletions or additions. In various embodiments, the derivative peptide comprises up to 3 amino acid substitutions, deletions or additions. In various embodiments, the derivative does not comprise a naturally occurring amino acid substitution, deletion or addition. In various embodiments, the derivative is not the D47N variant.

“Variant” and “mutant” when used herein in reference to the SHMOOSE peptide of the present invention refers a peptide having one or more naturally occurring amino acid substitutions, deletions or additions as compared to a “wild type” SHMOOSE peptide. For example, the variant “D47N,” is the version of the SHMOOSE peptide found in about 25% of Europeans, and this mutant/variant increases risk for AD by 30%.

“Modulation” or “modulates” or “modulating” as used herein refers to upregulation (i.e., activation or stimulation), down regulation (i.e., inhibition or suppression) of a response or the two in combination or apart.

“Pharmaceutically acceptable carriers” as used herein refer to conventional pharmaceutically acceptable carriers useful in this invention.

“Promote” and/or “promoting” as used herein refer to an augmentation in a particular behavior of a cell or organism.

“Subject” as used herein includes all animals, including mammals and other animals, including, but not limited to, companion animals, farm animals and zoo animals. The term “animal” can include any living multi-cellular vertebrate organisms, a category that includes, for example, a mammal, a bird, a simian, a dog, a cat, a horse, a cow, a rodent, and the like. Likewise, the term “mammal” includes both human and non-human mammals. In various embodiments, the subject is human.

“Therapeutically effective amount” as used herein refers to the quantity of a specified composition, or active agent in the composition, sufficient to achieve a desired effect in a subject being treated. A therapeutically effective amount may vary depending upon a variety of factors, including but not limited to the physiological condition of the subject (including age, sex, disease type and stage, general physical condition, responsiveness to a given dosage, desired clinical effect) and the route of administration. One skilled in the clinical and pharmacological arts will be able to determine a therapeutically effective amount through routine experimentation.

“Treat,” “treating” and “treatment” as used herein refer to both therapeutic treatment and prophylactic or preventative measures, wherein the object is to prevent or slow down (lessen) the targeted condition, disease or disorder (collectively “ailment”) even if the treatment is ultimately unsuccessful. Those in need of treatment may include those already with the ailment as well as those prone to have the ailment or those in whom the ailment is to be prevented.

Described herein are methods and compositions for treatment using novel mitochondrial peptides. Identified via genome wide scanning, mitochondrial single nucleotide polymorphism (SNP) mutations associated with the novel mitochondrial peptide are associated with neurodegeneration.

SHMOOSE (Small Human Mitochondrial Open reading frame Over the SErine-tRNA) is a newly discovered peptide that is implicated in neurodegenerative diseases. As such, artificial SHMOOSE peptide analogues could be used in prevention and treatment of these conditions. This invention includes the composition of matter of a family of peptide analogues of SHMOOSE, a newly discovered mitochondrial-derived peptide. The invention also includes antibodies and assays for the detection of the levels of the SHMOOSE peptide in the circulation and tissues of humans.

SHMOOSE is encoded by a small mitochondrial DNA open reading frame (ORF). SHMOOSE was detected in neuronal cells and CSF. Specifically, mass spectrometry and antibodies designed against the SHMOOSE reference sequences were used to detect SHMOOSE in nuclei and mitochondria of neuronal cells. SHMOOSE is a 58-amino acid peptide having a sequence of MPPCLTTWLSQLLKDNSYPLVLGPKNFGATPNKSNNHAH YYNHPNPDFPNSPHPYHPR (SEQ ID NO: 93) that contains an intrinsically disordered region. Mitochondrial-derived peptides (MDPs) are key factors in retrograde mitochondrial signaling as well as mitochondrial gene expression. Compared to the human nuclear genome, mitochondria have a modest sized circular genome of ˜16,570 bp, which ostensibly includes only 13 protein coding genes, which are all structural components of the electron transport chain system.

Mitochondrial DNA (mtDNA) replication and transcription starts are regulated by nuclear-encoded proteins and is thought to be transcribed as a single polycistronic precursor that is processed into individual genes by excising the strategically positioned 22 tRNAs (tRNA punctuation model), giving rise to two rRNAs and 13 mRNA.

The human mitochondrion has two promoters in the heavy strand (major and minor) in proximity, and one in the light strand, thereby giving rise to three different single polycistronic transcripts. The heavy major promoter is responsible for 80% of all mitochondrial RNA (mtRNA) transcripts. Although the entire gene is thought to be transcribed as a single transcript, the abundance of individual rRNA, tRNA, and mRNA transcripts varies greatly, and the rRNAs are the most abundant. This processing structure indicates an unexplored class of posttranscriptional processing in the mitochondria.

Importantly, many of the mRNA species identified from the mitochondria are discrete smaller length ones that do not map to the traditional mitochondrial protein encoding genes. For example, multiple such mRNAs are observed from the 16S rRNA. Parallel analysis of RNA ends (PARE) reveals a plethora of expected and unexpected cleavage sites have been discovered for the mitochondria. The majority of tRNAs and mRNA have distinct dominant cleavage sites at the 5′ termini, but intragenic cleavage sites are especially abundant in rRNAs. Notably, there is compelling evidence from the emerging field of small peptides showing biologically active peptides of 11-32 amino acids in length which are encoded by small open reading frames (sORFs) from a polycistronic mRNA.

Mitochondria are thought to have transferred their genome to the host nucleus leaving chromosomal “doppelgangers”, through the process of Nuclear Mitochondrial DNA-Transfer or nuclear insertions of mitochondrial origin (NUMTs). NUMTS come in various sizes from all parts of the mtDNA with various degrees of homology with the original sequences. Entire mtDNA can be found in the nuclear genome, although in most cases with substantial sequence degeneration. Most NUMTs are small insertions of <500 bp and only 12.85% are >1500 bp. The percentage identity is inversely correlated with size and the mean percentage between NUMTs and mtDNA is 79.2% with a range of 63.5% to 100% identity.

Described herein is a mitochondrial peptide. In one embodiment, the mitochondrial peptide includes a peptide with the amino acid sequence MPP CLT TWL SQL LKD NSY PLV LGP KNF GATP NKS NNH AHY YNH PNP DFP NSP HPY HPR (SEQ ID NO: 93), analog or derivative thereof.

In one embodiment, the mitochondrial peptide is 19-70 amino acids in length. In a particular embodiment, the mitochondrial peptide is 58 amino acids in length. In various embodiments, SHMOOSE analogs are 25 amino acids or about 25 amino acids in length. In various embodiments, SHMOOSE analogs are 20-30 amino acids in length; or 18-20, 20-22, 22-24, 24-26, 26-28, 28-30, or 30-32 amino acids in length. In one embodiment, the mitochondrial peptide includes a synthetic amino acid. In one embodiment, the mitochondrial peptide possesses less than about 25%, 25%, 30%, 35%, 40%, 45%, 50%, 55%, 60%, 65%, 70%, 75%, 80%, 85% or more percentage identity to MPP CLT TWL SQL LKD NSY PLV LGP KNF GATP NKS NNH AHY YNH PNP DFP NSP HPY HPR (SEQ ID NO: 93).

In various embodiments, peptides having a sequence in Table 1 are provided. In various embodiments, peptides are provided having a sequence identity of about 75%, 80%, 85%, 90%, 91%, 92%, 93%, 94%, 95%, 96%, 97%, 98%, 99% to any peptide in Table 1. In some embodiments, peptides are provided having a sequence identity of about 75%, 80%, 85%, 90%, 91%, 92%, 93%, 94%, 95%, 96%, 97%, 98%, 99% to a peptide of SEQ ID NO: 3 (PCLTTWLSQLLKDNSYPLVLGPKNF) in Table 1.

One of ordinary skill in the art can establish percentage identity according to methods known in the art, including establishing a comparison window between a reference amino acid sequence and a second amino sequence, to establish the degree of percentage identity.

In other embodiments, the mitochondrial peptide possesses a post-translational modification or other type of modification such as an artificial modification. In various embodiments, this includes for example, pegylation, fatty-acid conjugation lipidation, repeat polypeptide extension, IgG-Fc, CPT, HSA, ELP, transferrin, or albumin modification, among others. In various embodiments, these modifications can improve peptide stability, reduce enzyme degradation, increase half-life of the peptide, or increase cell permeability, as compared to a non-modified peptide. Described herein is a peptide. In various embodiments, the peptide is 19-70 amino acids in length. In various embodiments, the peptide is a recombinant peptide, or synthesized in a laboratory. In various embodiments, the peptide at position 1 (i.e., first N-terminal amino acid) is X1, position 2 is (X2) and so on (X3, X4, 5, X6, etc.), wherein X1, X2, X3, X4, X5, X6, etc. is selected from a group consisting of a natural or synthetic amino acid. In other embodiments, the mitochondrial peptide possesses a post-translational modification or other type of modification such as an artificial modification. In various embodiments, this includes for example, pegylation, fatty-acid conjugation lipidation, repeat polypeptide extension, IgG-Fc, CPT, HSA, ELP, transferrin, or albumin modification, among others. For example, modifications could include formylation, phosphorylation, acetylation at corresponding X1, X2, X3, X4, X5, X6, etc. positions in analogs or derivatives thereof. In various embodiments, the peptide possesses less than about 25%, 25%, 30%, 35%, 40%, 45%, 50%, 55%, 60%, 65%, 70%, 75%, 80%, 85% or more percentage identity to MPP CLT TWL SQL LKD NSY PLV LGP KNF GATP NKS NNH AHY YNH PNP DFP NSP HPY HPR (SEQ ID NO: 93).

In various embodiments, the peptide possesses at least 75%, 80%, 85%, 90%, 91%, 92%, 93%, 94%, 95%, 96%, 97%, 98%, 99% to a peptide of SEQ ID NO: 3 (PCLTTWLSQLLKDNSYPLVLGPKNF) in Table 1. In various embodiments, the peptide is 75%, 80%, 85% or more percentage identity to a portion of MPP CLT TWL SQL LKD NSY PLV LGP KNF GATP NKS NNH AHY YNH PNP DFP NSP HPY HPR (SEQ ID NO: 93), including for example, three or more, five or more, ten or more, fifteen or more, twenty or more, twenty-five or more amino acids of MPP CLT TWL SQL LKD NSY PLV LGP KNF GATP NKS NNH AHY YNH PNP DFP NSP HPY HPR (SEQ ID NO: 93), wherein the portion begins at X1, X2, X3, X4, etc. In some embodiments, the peptide has a sequence identity of at least 75%, 80%, 85%, 90%, 91%, 92%, 93%, 94%, 95%, 96%, 97%, 98%, 99% to a peptide of SEQ ID NOs: 3, 1, 15, 2, 4, 25, 98, 100, 103, 104, 105, or 107, wherein the portion begins at X1, X2, X3, X4, etc.

In various embodiments, the peptide is any one of those in Table 1.

Described herein is a method of increasing metabolic activating of cells in a subject in need thereof, using a mitochondrial peptide including administering a quantity of the mitochondrial peptide to a subject in need of treatment. In one embodiment, the mitochondrial peptide has a sequence of MPP CLT TWL SQL LKD NSY PLV LGP KNF GATP NKS NNH AHY YNH PNP DFP NSP HPY HPR (SEQ ID NO: 93), or analog or derivative thereof with 19-70 amino acids in length. In one embodiment, the mitochondrial peptide is 58 amino acids in length. In one embodiment, the mitochondrial-derived peptide is 25 amino acids in length or about 25 amino acids in length. In one embodiment, the mitochondrial-derived peptide is any one of those in Table 1. In one embodiment, the mitochondrial-derived peptide is a peptide of SEQ ID NO:3 in Table 1.

In various embodiments, the subject in need of increasing metabolic activating of cells include those who have a neurodegenerative disease. In various embodiments, the subject in need of increasing metabolic activating of cells include those who are at increased risk of having a neurodegenerative disease. In various embodiments, the subject in need of increasing metabolic activating of cells include those who are suspected of having or showing one or more symptoms of having a neurodegenerative disease. Neurodegenerative diseases include ALS, Parkinson's disease, Alzheimer's disease, Huntington disease, Prion disease, motor neuron diseases (MND), ataxias and palsys such as spinocerebellar ataxia (SCA), spinal muscular atrophy (SMA) and all other neurodegenerative diseases recognized in the art. In various embodiments, the aforementioned diseases include dominant mutant and sporadic forms, for example sporadic ALS, Alzheimer's and Parkinson's. In various embodiments, the disease or condition is Alzheimer's disease (AD). In various embodiments, the disease or condition is dementia. In various embodiments, the AD is Late-Onset Alzheimer's Disease (LOAD).

Described herein is a method of treating a disease and/or condition using a mitochondrial peptide including administering a quantity of the mitochondrial peptide to a subject in need of treatment. In one embodiment, the mitochondrial peptide is a sequence of MPP CLT TWL SQL LKD NSY PLV LGP KNF GATP NKS NNH AHY YNH PNP DFP NSP HPY HPR (SEQ ID NO: 93), analog or derivative thereof with 19-70 amino acids in length. In one embodiment, the mitochondrial peptide is 58 amino acids in length. In one embodiment, the mitochondrial-derived peptide is 25 amino acids in length or about 25 amino acids in length. In one embodiment, the mitochondrial-derived peptide is any one of those in Table 1. In one embodiment, the mitochondrial-derived peptide is a peptide of SEQ ID NO: 3 in Table 1. In various embodiments, the mitochondrial-derived peptide is a peptide having SEQ ID NOs: 3, 1, 15, 2, 4, 25, 98, 100, 103, 104, 105, or 107. In various embodiments, the method further includes selecting a subject in need of treatment prior to administering the peptide. Selection, for example, can be based on the expression level of the mitochondrial peptide or the presence or absence of SNPs as further described herein. In one embodiment, the quantity of the mitochondrial peptide administered is a therapeutically effective amount of the mitochondrial peptide. In one embodiment, the subject is a mammal. In one embodiment, the subject is a human.

In various embodiments, disease and/or condition suitable for treatment with the mitochondrial peptide or analogue composition described include neurodegenerative diseases. Neurodegenerative diseases include ALS, Parkinson's disease, Alzheimer's disease, Huntington disease, Prion disease, motor neuron diseases (MND), ataxias and palsys such as spinocerebellar ataxia (SCA), spinal muscular atrophy (SMA) and all other neurodegenerative diseases recognized in the art. In various embodiments, the aforementioned diseases include dominant mutant and sporadic forms, for example sporadic ALS, Alzheimer's and Parkinson's. In various embodiments, the disease or condition is Alzheimer's disease (AD). In various embodiments, the disease or condition is dementia. In various embodiments, the AD is Late-Onset Alzheimer's Disease (LOAD).

In various embodiments, the mitochondrial peptide increases tau in CSF. In various embodiments, the mitochondrial peptide reduces cognitive decline and/or reduces the rate of cognitive decline. In various embodiments, the mitochondrial peptide reduces the signs of disease in temporal cortex tissue or superior parietal lobe cortex tissue. In various embodiments, the mitochondrial peptide increases energy metabolism, by for example, optimizing fuel utilization in cells. In various embodiments, the subject is a carrier of the SNP 12372 (rs2853499).

In various embodiments, the subject does not express the peptide MPP CLT TWL SQL LKD NSY PLV LGP KNF GATP NKS NNH AHY YNH PNP DFP NSP HPY HPR (SEQ ID NO: 93). In various embodiments, the subject expresses low amounts of the peptide MPP CLT TWL SQL LKD NSY PLV LGP KNF GATP NKS NNH AHY YNH PNP DFP NSP HPY HPR (SEQ ID NO: 93) relative to a healthy normal subject. In other embodiments, the subject possesses a metabolic signature of low SHMOOSE activity. In other embodiments, the subject possesses a metabolic signature of high or aberrant SHMOOSE activity. In various embodiments, the subject is administered a dominant negative analog and/or derivative of SHMOOSE.

In various embodiments, the compositions according to the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

In various embodiments, the peptide or composition is administered via intracerebroventricular injection.

The compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. In some embodiments, the pharmaceutically acceptable carrier also serves as a preservative or stabilizer for the peptide, and/or reduce degradation of the peptide. As such, the composition comprising the peptide and carrier will have a longer “shelf life” than a composition comprising the peptide without the carrier. In various embodiments, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Described herein is a method of diagnosing an individual for a disease and/or condition. In various embodiments, the method includes selecting a subject, detecting the presence, absence, or expression level of one or more biomarkers, and diagnosing the subject for a disease and/or condition, based on the presence, absence, or expression level of the one or more biomarkers. In various embodiments, the biomarker includes a mitochondrial peptide. In various embodiments, the biomarker includes MPP CLT TWL SQL LKD NSY PLV LGP KNF GATP NKS NNH AHY YNH PNP DFP NSP HPY HPR (SEQ ID NO: 93). For example, the subject may be diagnosed if expressing a low, high, or aberrant amount of the peptide MPP CLT TWL SQL LKD NSY PLV LGP KNF GATP NKS NNH AHY YNH PNP DFP NSP HPY HPR (SEQ ID NO: 93) relative to a healthy normal subject. In various embodiments, detection of the presence, absence, or expression level of the biomarker includes antibody detection of the one of or more biomarkers, including the use of, for example, a monoclonal antibody, polyclonal antibody, antisera, other immunogenic detection, and mass spectrometry detection methods.

In another embodiment, the biomarker includes a single nucleotide polymorphism (SNP). In various embodiments, the SNP is 12372 (rs2853499). One of ordinary skill in the art is apprised of the methods capable of SNP detection.

In various embodiments, disease and/or condition suitable for treatment with the mitochondrial peptide or analogue composition described include neurodegenerative diseases. Neurodegenerative diseases include amyotrophic lateral sclerosis (ALS), Parkinson's disease, Alzheimer's disease, Huntington disease, Prion disease, motor neuron diseases (MND), ataxias and palsys such as spinocerebellar ataxia (SCA), spinal muscular atrophy (SMA) and all other neurodegenerative diseases recognized in the art. In various embodiments, the aforementioned diseases include dominant mutant and sporadic forms, for example sporadic ALS, Alzheimer's and Parkinson's. In particular embodiments, the disease and/or condition suitable for treatment with the mitochondrial peptide or analogue composition is Alzheimer's disease.

Described herein is a method of detecting one or more biomarkers. In various embodiments, the method includes detecting the presence, absence, or expression level of one or more biomarkers in a biological sample obtained from a subject desiring a determination regarding the one or more biomarkers; and detecting the presence, absence, or expression level of the one or more biomarkers. In various embodiments, the one or more biomarkers includes a peptide of the sequence MPPCLTTWLSQLLKDNSYPLVLGPKNFGATPNKSNNHAHYY NHPNPDFPNSPHPYHPR (SEQ ID NO: 93), MPPCLTTWLSQLLKDNSYPLVLGPKNFG ATPNKSNNHAHYYNHPNPNFPNSPHPYHPR (SEQ ID NO: 94), or a single nucleotide polymorphism (SNP) rs2853499. For example, detecting the presence, absence, or expression level includes an immunoassay. In various embodiments, the one or more biomarkers includes a single nucleotide polymorphism (SNP) rs2853499, wherein an “A” allele is at the SNP position. In various embodiments, the method further includes diagnosing the subject with a disease and/or condition or with an increased likelihood of having the disease and/or condition when the presence of the peptide having SEQ ID NO:94 is detected, OR diagnosing the subject with a disease and/or condition or with an increased likelihood of having the disease and/or condition when low expression levels of the peptide having SEQ ID NO:93, as compared to a healthy control, is detected, OR diagnosing the subject with a disease and/or condition when a single nucleotide polymorphism (SNP) rs2853499, wherein an “A” allele is at the SNP position, is detected. For example, the disease and/or condition is a neurodegenerative disease and/or condition. In various embodiments, the neurodegenerative disease and/or condition is selected from the group consisting of Alzheimer's disease, Parkinson's disease, dementia, and combinations thereof.

Described herein is a method of detecting a genotype of a mitochondrial-derived peptide in a subject in need thereof. In various embodiments, the method includes assaying a biological sample obtained from the subject to detect genotype at a single nucleotide polymorphism (SNP). For example, the SNP is rs2853499. In various embodiments, the method further includes detecting an A allele at the SNP in the subject. For example, the subject has Alzheimer's disease or has a risk factor for developing Alzheimer's disease. In various embodiments, the detection detects a count of the A allele at the SNP higher than that in a control subject, in the subject having Alzheimer's disease or has a risk factor for developing Alzheimer's disease. In various embodiments, the method further includes administering an Alzheimer's disease therapy to the subject. In various embodiments, the subject desires a determination regarding a neurodegeneration disease or disorder, optionally Alzheimer's disease.

The present invention further provides a method of enhancing efficacy of a treatment disease and/or condition using a mitochondrial peptide, including the steps of selecting a subject in need of treatment, and administering a quantity of the mitochondrial peptide to a subject receiving treatment a disease and/or condition, wherein the mitochondrial peptide enhancing the efficacy of the disease and/or condition, thereby enhancing efficacy of the treatment. In one embodiment, the mitochondrial peptide is administered simultaneously with a composition capable of treating an inflammatory disease and/or condition. In one embodiment, the mitochondrial peptide is administered sequentially, before or after administration, of a composition capable of treating a disease and/or condition. In one embodiment, the subject is a human. For example, the mitochondrial peptides and analog compositions of the invention can be co-administered with other therapeutic agents for the treatment of neurodegenerative diseases. Co-administration can be simultaneous, e.g., in a single pharmaceutical composition or separate compositions. The compositions of the invention can also be administered separately from the other therapeutic agent(s), e.g., on an independent dosing schedule.

In various embodiments, the present invention further provides a pharmaceutical composition. In one embodiment, the pharmaceutical composition includes a mitochondrial peptide and a pharmaceutically acceptable carrier. In one embodiment, the sequence of the mitochondrial peptide is MPP CLT TWL SQL LKD NSY PLV LGP KNF GATP NKS NNH AHY YNH PNP DFP NSP HPY HPR (SEQ ID NO: 93). In various embodiments, the peptide is 75%, 80%, 85% or more percentage identity to a portion of MPP CLT TWL SQL LKD NSY PLV LGP KNF GATP NKS NNH AHY YNH PNP DFP NSP HPY HPR (SEQ ID NO: 93), including for example, three or more, five or more, ten or more, fifteen or more, twenty or more, twenty-five or more amino acids of MPP CLT TWL SQL LKD NSY PLV LGP KNF GATP NKS NNH AHY YNH PNP DFP NSP HPY HPR (SEQ ID NO: 93), wherein the portion begins at X1, X2, X3, X4, etc. In various embodiments, the peptide is 75%, 80%, 85% or more percentage identity to one or more of those peptides in Table 1. In various embodiments, the peptide is 75%, 80%, 85% or more percentage identity to SEQ ID NO: 3 in Table 1. In some embodiments, the peptide has a sequence identity of at least 75%, 80%, 85%, 90%, 91%, 92%, 93%, 94%, 95%, 96%, 97%, 98%, 99% to a peptide of SEQ ID NOs: 3, 1, 15, 2, 4, 25, 98, 100, 103, 104, 105, or 107.

In some embodiments, the bioactive mitochondrial peptide is as small as 6-9 amino-acids, as well as some that are 19-70 amino acids in length. In one embodiment, the mitochondrial peptide is 58 amino acids in length. In one embodiment, the mitochondrial peptide is 25 amino acids in length, or about 25 amino acids in length. In one embodiment, the mitochondrial peptide in the pharmaceutical composition includes a therapeutically effective amount of the mitochondrial peptide. In one embodiment, pharmaceutical composition includes one or more mitochondrial peptides and a pharmaceutically acceptable carrier.

In various embodiments, the present invention further provides a method of manufacturing a mitochondrial peptide. In one embodiment, the method of manufacturing includes the steps of providing one or more polynucleotides encoding a mitochondrial peptide, expressing the one or more polynucleotides in a host cell, and extracting the mitochondrial peptide from the host cell. In one embodiment, the method of manufacturing includes the steps of expressing the one or more polynucleotides in a host cell, and extracting the mitochondrial peptide from the host cell. In one embodiment, the one or more polynucleotides are a sequence encoding MPP CLT TWL SQL LKD NSY PLV LGP KNF GATP NKS NNH AHY YNH PNP DFP NSP HPY HPR (SEQ ID NO: 93), or a mitochondrial peptide possessing more than about 20%, 25%, 30%, 35%, 40%, 45%, 50%, 55%, 60%, 65%, 70%, 75%, 80%, 85% or more percentage identity to MPP CLT TWL SQL LKD NSY PLV LGP KNF GATP NKS NNH AHY YNH PNP DFP NSP HPY HPR (SEQ ID NO: 93). In various embodiments, the peptide has 75%, 80%, 85% or more percentage identity to a portion of MPP CLT TWL SQL LKD NSY PLV LGP KNF GATP NKS NNH AHY YNH PNP DFP NSP HPY HPR (SEQ ID NO: 93), including for example, three or more, five or more, ten or more, fifteen or more, twenty or more, twenty-five or more amino acids of MPP CLT TWL SQL LKD NSY PLV LGP KNF GATP NKS NNH AHY YNH PNP DFP NSP HPY HPR (SEQ ID NO: 93), wherein the portion begins at X1, X2, X3, X4, etc.

In various embodiments, the peptide has at least 75%, 80%, 85%, 90%, 91%, 92%, 93%, 94%, 95%, 96%, 97%, 98%, or 99% identity to SEQ ID NO: 3 in Table 1. In various embodiments, the peptide has at least 75%, 80%, 85%, 90%, 91%, 92%, 93%, 94%, 95%, 96%, 97%, 98%, or 99% identity to SEQ ID NO: 1 in Table 1. In various embodiments, the peptide has at least 75%, 80%, 85%, 90%, 91%, 92%, 93%, 94%, 95%, 96%, 97%, 98%, or 99% identity to SEQ ID NO: 15 in Table 1. In various embodiments, the peptide has at least 75%, 80%, 85%, 90%, 91%, 92%, 93%, 94%, 95%, 96%, 97%, 98%, or 99% identity to SEQ ID NO: 2 in Table 1. In various embodiments, the peptide has at least 75%, 80%, 85%, 90%, 91%, 92%, 93%, 94%, 95%, 96%, 97%, 98%, or 99% identity to SEQ ID NO: 4 in Table 1.

In another embodiment, the method of manufacturing includes the steps of peptide synthesis using liquid-phase synthesis or solid-phase synthesis. In one embodiment, the solid-phase synthesis is Fmoc or BOC synthesis.

TABLE 1
SHMOOSE analogues/derivatives and their efficacy, measured by MTT, as a
percentage compared to SHMOOSE (SHMOOSE activity as 100%).

| SEQ | | | | | |
|---|---|---|---|---|---|
| ID | | % | Standard | Standard | Confidence |
| NO: | Sequence | Efficacy | Deviation | Error | Interval |
| 3 | PCLTTWLSQLLKDNSYPLVLGPKNF | 115.35 | 87.55 | 50.55 | 217.48 |
| 1 | MPPCLTTWLSQLLKDNSYPLVLGPK | 62.23 | 128.92 | 74.43 | 320.26 |
| 15 | DNSYPLVLGPKNFGATPNKSNNHAH | 51.51 | 40.95 | 23.65 | 101.74 |
| 2 | PPCLTTWLSQLLKDNSYPLVLGPKN | 37.99 | 85.27 | 49.23 | 211.83 |
| 4 | CLTTWLSQLLKDNSYPLVLGPKNFG | 12.47 | 48.16 | 27.81 | 119.64 |
| 35 | GPKNFGATPNKSNNHAHYYNHPNPN | 8.68 | 139.88 | 80.76 | 347.48 |
| 57 | QLLKDNDDPLVLGPKNFGADPNKDN | −1.36 | 105.44 | 60.88 | 261.93 |
| 8 | WLSQLLKDNSYPLVLGPKNFGATPN | −3.65 | 106.77 | 61.64 | 265.23 |
| 25 | KNFGATPNKSNNHAHYYNHPNPDFP | −8.52 | 124.05 | 71.62 | 308.17 |
| 5 | LTTWLSQLLKDNSYPLVLGPKNFGA | −13.61 | 75.39 | 43.53 | 187.28 |
| 72 | NFGADPNKDNNHAHDDNHPNPDFPN | −28.57 | 101.63 | 58.67 | 252.45 |
| 55 | LDQLLKDNDDPLVLGPKNFGADPNK | −38.64 | 154.46 | 89.17 | 383.69 |
| 52 | DDWLDQLLKDNDDPLVLGPKNFGAD | −38.98 | 370.41 | 213.86 | 920.15 |
| 6 | TTWLSQLLKDNSYPLVLGPKNFGAT | −42.74 | 146.52 | 84.59 | 363.98 |
| 54 | WLDQLLKDNDDPLVLGPKNFGADPN | −45.99 | 258.73 | 149.38 | 642.71 |
| 21 | VLGPKNFGATPNKSNNHAHYYNHPN | −50.24 | 88.16 | 50.90 | 218.99 |
| 58 | LLKDNDDPLVLGPKNFGADPNKDNN | −58.59 | 180.15 | 104.01 | 447.51 |
| 13 | LKDNSYPLVLGPKNFGA | −58.60 | 43.86 | 25.32 | 108.96 |
| | TPNKSNNH | | | | |
| 46 | SNNHAHYYNHPNPNFPNSPHPYHPR | −59.27 | 180.62 | 104.28 | 448.68 |
| 86 | GADPNKDNNHAHDDNHPNPNFPNDP | −62.75 | 21.97 | 12.69 | 54.59 |
| 53 | DWLDQLLKDNDDPLVLGPKNFGADP | −76.54 | 142.46 | 82.25 | 353.89 |
| 16 | NSYPLVLGPKNFGATPNKSNNHAHY | −79.03 | 145.50 | 84.01 | 361.45 |
| 9 | LSQLLKDNSYPLVLGPKNFGATPNK | −82.17 | 1.01 | 0.58 | 2.50 |
| 88 | DPNKDNNHAHDDNHPNPNFPNDPHP | −84.19 | 44.43 | 25.65 | 110.37 |
| 84 | NFGADPNKDNNHAHDDNHPNPNFPN | −84.90 | 74.12 | 42.79 | 184.13 |
| 11 | QLLKDNSYPLVLGPKNFGATPNKSN | −85.19 | 39.49 | 22.80 | 98.10 |
| 92 | DNNHAHDDNHPNPNFPNDPHPDHPR | −85.47 | 79.56 | 45.94 | 197.65 |
| 56 | DQLLKDNDDPLVLGPKNFGADPNKD | −93.30 | 154.74 | 89.34 | 384.40 |
| 12 | LLKDNSYPLVLGPKNFGATPNKSNN | −97.85 | 135.18 | 78.05 | 335.81 |
| 91 | KDNNHAHDDNHPNPNFPNDPHPDHP | −104.32 | 151.75 | 87.61 | 376.98 |
| 10 | SQLLKDNSYPLVLGPKNFGATPNKS | −107.19 | 84.05 | 48.52 | 208.78 |
| 7 | TWLSQLLKDNSYPLVLGPKNFGATP | −113.24 | 63.14 | 36.46 | 156.86 |
| 71 | KNFGADPNKDNNHAHDDNHPNPDFP | −113.32 | 104.70 | 60.45 | 260.08 |
| 33 | KSNNHAHYYNHPNPDFPNSPHPYHP | −119.66 | 69.36 | 40.05 | 172.30 |
| 36 | PKNFGATPNKSNNHAHYYNHPNPNF | −120.40 | 13.67 | 7.89 | 33.96 |
| 51 | LDDWLDQLLKDNDDPLVLGPKNFGA | −132.69 | 489.64 | 282.70 | 1216.34 |
| 18 | YPLVLGPKNFGATPNKSNNHAHYYN | −133.09 | 94.84 | 54.76 | 235.59 |
| 22 | LGPKNFGATPNKSNNHAHYYNHPNP | −140.81 | 44.00 | 25.40 | 109.29 |
| 68 | LGPKNFGADPNKDNNHAHDDNHPNP | −144.36 | 141.37 | 81.62 | 351.18 |
| 32 | NKSNNHAHYYNHPNPDFPNSPHPYH | −145.56 | 77.80 | 44.92 | 193.27 |
| 82 | PKNFGADPNKDNNHAHDDNHPNPNF | −146.18 | 147.23 | 85.00 | 365.73 |
| 60 | KDNDDPLVLGPKNFGADPNKDNNHA | −146.91 | 106.31 | 61.38 | 264.08 |
| 14 | KDNSYPLVLGPKNFGATPNKSNNHA | −147.96 | 32.29 | 18.64 | 80.21 |
| 47 | MPPCLDDWLDQLLKDNDDPLVLGPK | −150.17 | 19.60 | 11.32 | 48.69 |
| 75 | ADPNKDNNHAHDDNHPNPDFPNDPH | −157.10 | 143.07 | 82.60 | 355.41 |
| 70 | PKNFGADPNKDNNHAHDDNHPNPDF | −157.35 | 11.45 | 6.61 | 28.45 |
| 66 | LVLGPKNFGADPNKDNNHAHDDNHP | −157.61 | 84.88 | 49.01 | 210.86 |
| 48 | PPCLDDWLDQLLKDNDDPLVLGPKN | −159.22 | 141.14 | 81.49 | 350.62 |
| 65 | PLVLGPKNFGADPNKDNNHAHDDNH | −163.31 | 163.61 | 94.46 | 406.44 |
| 49 | PCLDDWLDQLLKDNDDPLVLGPKNF | −168.90 | 199.23 | 115.03 | 494.93 |
| 81 | GPKNFGADPNKDNNHAHDDNHPNPN | −172.67 | 263.90 | 152.36 | 655.55 |
| 79 | KDNNHAHDDNHPNPDFPNDPHPDHP | −183.31 | 71.20 | 41.11 | 176.87 |
| 69 | GPKNFGADPNKDNNHAHDDNHPNPD | −184.35 | 50.22 | 28.99 | 124.75 |
| 76 | DPNKDNNHAHDDNHPNPDFPNDPHP | −184.63 | 110.40 | 63.74 | 274.25 |
| 24 | PKNFGATPNKSNNHAHYYNHPNPDF | −185.28 | 33.65 | 19.43 | 83.59 |
| 41 | ATPNKSNNHAHYYNHPNPNFPNSPH | −188.45 | 72.83 | 42.05 | 180.92 |
| 19 | PLVLGPKNFGATPNKSNNHAHYYNH | −191.36 | 43.92 | 25.36 | 109.11 |
| 17 | SYPLVLGPKNFGATPNKSNNHAHYY | −195.89 | 26.25 | 15.16 | 65.21 |
| 34 | SNNHAHYYNHPNPDFPNSPHPYHPR | −195.91 | 54.15 | 31.26 | 134.51 |
| 38 | NFGATPNKSNNHAHYYNHPNPNFPN | −196.67 | 39.05 | 22.54 | 97.00 |
| 23 | GPKNFGATPNKSNNHAHYYNHPNPD | −210.91 | 82.02 | 47.36 | 203.76 |
| 80 | DNNHAHDDNHPNPDFPNDPHPDHPR | −214.13 | 53.74 | 31.03 | 133.51 |
| 63 | DDPLVLGPKNFGADPNKDNNHAHDD | −216.49 | 145.96 | 84.27 | 362.59 |
| 27 | FGATPNKSNNHAHYYNHPNPDFPNS | −226.16 | 39.99 | 23.09 | 99.34 |
| 90 | NKDNNHAHDDNHPNPNFPNDPHPDH | −226.81 | 104.44 | 60.30 | 259.45 |
| 37 | KNFGATPNKSNNHAHYYNHPNPNFP | −227.50 | 62.29 | 35.96 | 154.74 |
| 74 | GADPNKDNNHAHDDNHPNPDFPNDP | −228.29 | 51.79 | 29.90 | 128.65 |
| 28 | GATPNKSNNHAHYYNHPNPDFPNSP | −229.42 | 15.85 | 9.15 | 39.37 |
| 67 | VLGPKNFGADPNKDNNHAHDDNHPN | −231.08 | 77.02 | 44.47 | 191.32 |
| 26 | NFGATPNKSNNHAHYYNHPNPDFPN | −232.44 | 80.84 | 46.67 | 200.82 |
| 78 | NKDNNHAHDDNHPNPDFPNDPHPDH | −240.90 | 70.14 | 40.49 | 174.23 |
| 20 | LVLGPKNFGATPNKSNNHAHYYNHP | −248.54 | 97.29 | 56.17 | 241.68 |
| 62 | NDDPLVLGPKNFGADPNKDNNHAHD | −250.35 | 131.56 | 75.96 | 326.81 |
| 64 | DPLVLGPKNFGADPNKDNNHAHDDN | −254.41 | 92.13 | 53.19 | 228.87 |
| 29 | ATPNKSNNHAHYYNHPNPDFPNSPH | −262.37 | 128.70 | 74.30 | 319.70 |
| 45 | KSNNHAHYYNHPNPNFPNSPHPYHP | −292.14 | 119.44 | 68.96 | 296.71 |
| 40 | GATPNKSNNHAHYYNHPNPNFPNSP | −302.11 | 108.87 | 62.86 | 270.46 |
| 73 | FGADPNKDNNHAHDDNHPNPDFPND | −306.02 | 162.21 | 93.65 | 402.96 |
| 50 | CLDDWLDQLLKDNDDPLVLGPKNFG | −340.42 | 37.64 | 21.73 | 93.50 |
| 30 | TPNKSNNHAHYYNHPNPDFPNSPHP | −367.82 | 193.82 | 111.90 | 481.47 |
| 42 | TPNKSNNHAHYYNHPNPNFPNSPHP | −392.68 | 148.12 | 85.52 | 367.95 |
| 85 | FGADPNKDNNHAHDDNHPNPNFPND | −476.83 | 102.08 | 58.93 | 253.57 |
| 31 | PNKSNNHAHYYNHPNPDFPNSPHPY | −477.36 | 2.22 | 1.28 | 5.52 |
| 59 | LKDNDDPLVLGPKNFGADPNKDNNH | −528.69 | 87.91 | 50.75 | 218.38 |
| 77 | PNKDNNHAHDDNHPNPDFPNDPHPD | −543.17 | 79.01 | 45.62 | 196.28 |
| 83 | KNFGADPNKDNNHAHDDNHPNPNFP | −649.91 | 138.03 | 79.69 | 342.88 |
| 87 | ADPNKDNNHAHDDNHPNPNFPNDPH | −740.82 | 110.13 | 63.58 | 273.57 |
| 39 | FGATPNKSNNHAHYYNHPNPNFPNS | −759.10 | 102.38 | 59.11 | 254.32 |
| 43 | PNKSNNHAHYYNHPNPNFPNSPHPY | −855.87 | 60.82 | 35.11 | 151.08 |
| 61 | DNDDPLVLGPKNFGADPNKDNNHAH | −937.12 | 178.16 | 102.86 | 442.57 |
| 89 | PNKDNNHAHDDNHPNPNFPNDPHPD | −1089.83 | 16.84 | 9.73 | 41.85 |
| 44 | NKSNNHAHYYNHPNPNFPNSPHPYH | −1177.02 | 58.37 | 33.70 | 145.00 |
| 97 | MPPCITTWLSQLLKDNSYPLVLGPKNFGA | −1.709402 | 3.916731 | 2.261326 | 9.7297 |
| | TPNKSNNHAHYYNHPNPDFPNSPHPYHP | | | | |
| | R | | | | |
| 98 | MPPCITTWISQLLKDNSYPLVLGPKNFGA | 1.709402 | 14.803853 | 8.547009 | 36.77481 |
| | TPNKSNNHAHYYNHPNPDFPNSPHPYHP | | | | |
| | R | | | | |
| 99 | MPPCITTWISQILKDNSYPLVLGPKNFGA | −7.692308 | 16.012815 | 9.245003 | 39.77804 |
| | TPNKSNNHA | | | | |
| | HYYNHPNPDFPNSPHPYHPR | | | | |
| 100 | MPPCITTWISQIIKDNSYPLVLGPKNFGAT | 4.273504 | 16.284238 | 9.401709 | 40.45229 |
| | PNKSNNHAHYYNHPNPDFPNSPHPYHPR | | | | |
| 101 | MPPCLTTWISQIIKDNSYPLVLGPKNFGA | −10.25641 | 17.948718 | 10.362697 | 44.58709 |
| | TPNKSNNHA | | | | |
| | HYYNHPNPDFPNSPHPYHPR | | | | |
| 102 | MPPCLTTWLSQIIKDNSYPLVLGPKNFGA | −25.641026 | 17.764624 | 10.25641 | 44.12977 |
| | TPNKSNNHAHYYNHPNPDFPNSPHPYHP | | | | |
| | R | | | | |
| 103 | MPPCLTTWLSQLIKDNSYPLVLGPKNFGA | 86.324786 | 57.697056 | 33.311411 | 143.32743 |
| | TPNKSNNHAHYYNHPNPDFPNSPHPYHP | | | | |
| | R | | | | |
| 104 | MPSCLTTWLSQLLKDNSYPLVLGPKNFG | 82.051282 | 119.91121 | 69.230769 | 297.87596 |
| | ATPNKSNNHAHYYNHPNPDFPNSPHPYH | | | | |
| | PR | | | | |
| 105 | MPSCLTTWLSSLLKDNSYPLVLGPKNFG | 44.444444 | 36.652618 | 21.161399 | 91.05015 |
| | ATPNKSNNHAHYYNHPNPDFPNSPHPYH | | | | |
| | PR | | | | |
| 106 | MPSCLTTWLSSLLSDNSYPLVLGPKNFGA | −14.529915 | 19.245009 | 11.111111 | 47.80725 |
| | TPNKSNNHAHYYNHPNPDFPNSPHPYHP | | | | |
| | R | | | | |
| 107 | MPSCLTTWISSIISDNSYPLVLGPKNFGAT | 134.188034 | 42.158504 | 24.340224 | 104.72753 |
| | PNKSNNHAHYYNHPNPDFPNSPHPYHPR | | | | |

### EXAMPLES

Described herein are non-limiting examples of the claimed invention.

### Example 1. SHMOOSE (Small Human Mitochondrial Open Reading Frame Over the SErine-(RNA) Peptide

SHMOOSE is a newly discovered peptide of sequence MPP CLT TWL SQL LKD NSY PLV LGP KNF GATP NKS NNH AHY YNH PNP DFP NSP HPY HPR (SEQ ID NO: 93), whose dysfunctional mutation and dysregulation, is implicated in neurodegenerative diseases. As such, artificial SHMOOSE peptide analogues could be used in prevention and treatment of these conditions. Described herein is SHMOOSE role in neurodegenerative diseases, including Alzheimer's disease (AD), peptide analogues of SHMOOSE, including their role in energy metabolism.

### Example 2. Properties of SHMOOSE

SHMOOSE is encoded in an opening reading frame discovered using a mitochondrial genome-wide association study. Studies have correlated a SNP that changes the SHMOOSE sequence and increases risk for Alzheimer's disease pathology and cognition. This polymorphism is highly prevalent in people of European ancestry.

More specifically, overexpression of SHMOOSE increases neuronal-type cell survival. Interestingly a SNP was identified that causes SHMOOSE to act less potently in neuronal-type cells. SHMOOSE 12372 SNP (rs2853499) with A nucleotide was associated with increased decrease in cognitive scores compared to wild-type G nucleotide SHMOOSE.

### Example 3. Role of SHMOOSE in Cellular Metabolization

Without being bound by any hypothesis, SHMOOSE optimizes fuel utilization and localizes in the mitochondria and nuclei. Based on this role, the SHMOOSE role in cellular functions can be exploited by developing potent SHMOOSE analogues, including for the purpose of treating neurodegenerative diseases such as Alzheimer's and Parkinson's in cellular models and animal models.

### Example 4. SHMOOSE Expression, Preliminary Cell Biology Investigations

To decipher the role of SHMOOSE, preliminary studies confirmed that SHMOOSE can be expressed and synthesized for administration to cells, including the mutant D47N subtype. Upon administration to cells, it was observed that SHMOOSE improves neuronal-cell viability in amyloid beta toxic assays. In contrast, mutant D47N SHMOOSE did not improve neuronal-cell viability.

Further exploring the potential roles of SHMOOSE in apparent neuronal cell viability and in view of mitochondria in cell metabolism, energy map measurement revealed that SHMOOSE optimizes fuel utilization in cells. This functional observation of fuel utilization was confirmed by observations that SHMOOSE localizes in mitochondria. Preliminary structural analysis indicated that SHMOOSE has 7 high confidence phosphorylation sites.

### Example 5. Dysfunction and Dysregulation of SHMOOSE Demonstrate Broad Effects in Neurodegeneration

To further investigate the role of SHMOOSE in disease pathology, including neurodegenerative diseases, further investigation confirm that SHMOOSE genotype predicts Alzheimer's Disease (AD) phenotypes and interacts with nuclear genotypes, A SNP 12372 (rs2853499) in SHMOOSE predicts AD, cognition, brain structure, and brain gene expression.

Interestingly, the above interactions further indicated that SHMOOSE mtSNP predicts thinner hippocampi—a crucial brain region involved in AD. Interestingly, SHMOOSE mtSNP was also associated greater temporal pole volume—a feature commonly seen in AD. SHMOOSE expression is higher in AD and especially higher in AD for SHMOOSE mtSNP carriers. These results are strongly suggestive that SHMOOSE possesses broader roles in neuronal function, with dysfunction and dysregulation of SHMOOSE leading to neurodegeneration,

Affirming the broader effects of SHMOOSE dysfunction, SHMOOSE genotype associates with dramatic temporal cortical gene expression differences. These results demonstrate that SHMOOSE mtSNP carriers have differentially expressed genes that relate to RNA processing in the nucleus, metabolic processing in the mitochondria, and additional extracellular activity.

### Example 6. Metabolomics and Neurodegeneration

To elucidate the relationship between an apparent role for SHMOOSE in homeostasis and energy processing, and dysfunctional SHMOOSE leading to neurodegeneration, additional studies investigating cell biology and disease state were explored.

The Inventors observed that SHMOOSE is expressed in neurons. Specifically, endogenous SHMOOSE is found in the nucleus and in the mitochondria. Custom antibody against SHMOOSE peptides (amino acids 32-58) demonstrated that SHMOOSE, including artificial SHMOOSE peptides, can be internalized inside the cell and goes to the mitochondria and nucleus.

Connecting the functional observation of energy processing and localization in mitochondria, SHMOOSE may protect from Aβ toxicity by improving mitochondrial-derived energy generation capacity. SHMOOSE protects from Aβ toxicity in neurons. SNP12372 (rs2853499) leads to a D47N mutant that does not protect neurons from Aβ toxicity. These results are affirmed by observations that SHMOOSE increases MTT signal in neurons. MTT is a readout that captures NAD(P)H flux, which represents metabolism. SHMOOSE, including artificial SHMOOSE peptides, boosts mitochondrial function and spare capacity in neurons.

Gene enrichment analysis of SHMOOSE. SHMOOSE changes gene expression related to lipid synthesis and mitochondrial function; mutant SHMOOSE amplifies signature; this matches human association data. SHMOOSE turns on and off genes related to RNA processing and lipid metabolism. It also has profound effects on mitochondrial gene expression. D47N amplifies these effects because these cells make wild type, natural SHMOOSE.

### Example 7. Animal Studies

SHMOOSE attenuates weight gain in mice; improves liver enzymes. The Inventors fed mice a high fat diet for 2 weeks and found that: SHMOOSE IP injected mice did not gain as much weight; SHMOOSE goes into the blood stream and has a strong half-life; SHMOOSE decreases AST and ALT, enzymes of which are involved in liver metabolism. Lower levels suggest protection against high fat diet.

### Example 8. SHMOOSE Analogues

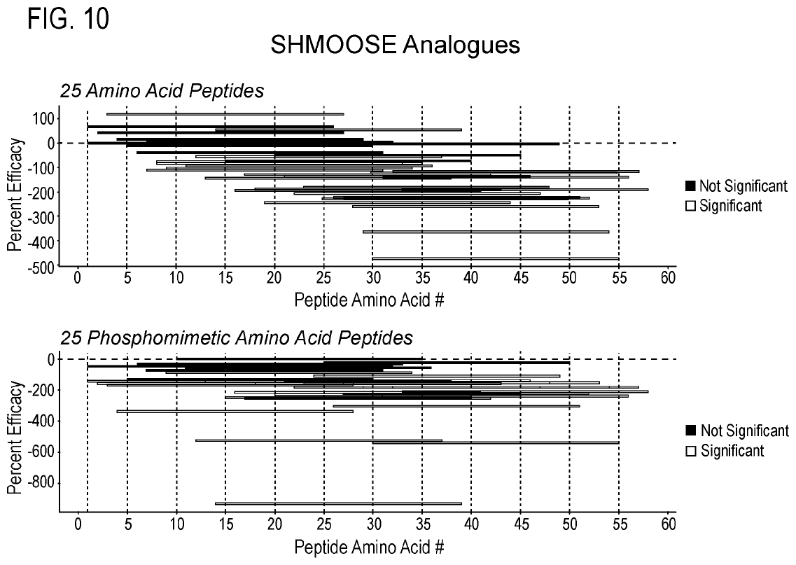

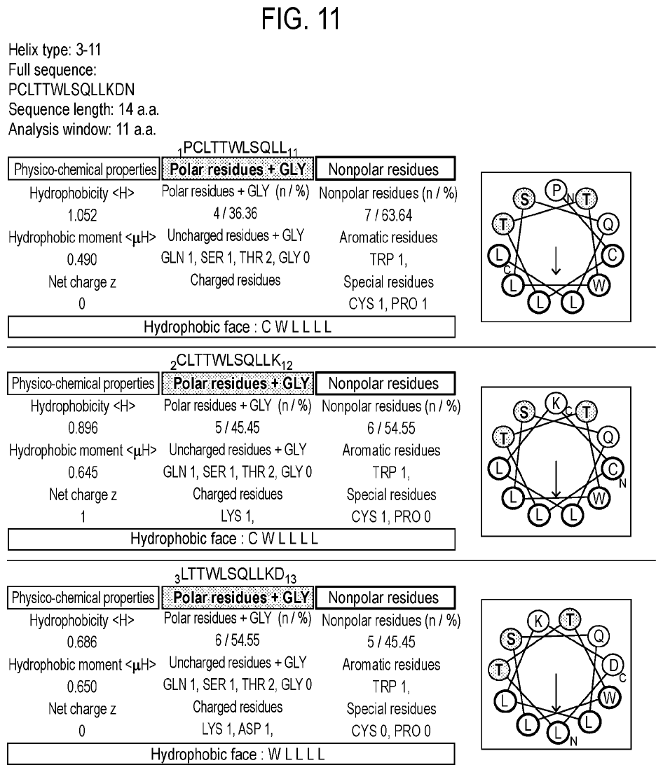

The Inventors made 103 analogues (Table 1). Referring to FIG. 10, in the top figure, each bar represents a 25-amino acid fragment of the SHMOOSE peptide. The Inventors found that two peptides have greater activity than SHMOOSE, as measured by MTT. In FIG. 10, the bottom figure shows that pseudophosphorylation of these analogues turns off the activity. FIG. 11 shows an exemplary analogue of the SHMOOSE peptide having a sequence: PCLTTWLSQLLKDN (SEQ ID NO: 95). The sequence length of the analogue is 14 amino acids and 3 different analysis windows of the analogue, each having 11 amino acids, were characterized.

### Example 9. Structural Features of SHMOOSE

Homology studies suggest the first 15 amino acid residues of SHMOOSE as related to protein sequences related to mitochondrial localization and nuclear export. This N-terminal region is more highly conserved in mammals, including the 26 amino acids possessing conservation in mouse.

By contrast, the C-terminal region of up to 30 amino acids, possess little or no homology to readily identifiable mammalian peptides or proteins. This reflects the bacterial origin of mitochondria in multicellular organisms.

SHMOOSE co-expression signature in human brain is shown with genes related to nuclear and mitochondrial ribosomal organization.

A SNP (rs2853499) in the SHMOOSE ORF associates with neurodegeneration. Rs2853499 predicts Alzheimer's; it also predicts human brain temporal cortex anatomy (UK Biobank; n=20,000).

Transcriptome signature analysis identified differentially expressed genes in SHMOOSE mtSNP carriers, cognitively normal individuals (n=78), which shows that SHMOOSE not only co-expresses with mitochondrial genes, but it also differentiates ribosomal biogenesis gene expression.

SHMOOSE pharmacokinetics was studied, where 2.5 mg SHMOOSE/kg in 25-week old C57/BI6 mice were intraperitoneally injected.

Further studies include performing binding partner assays, via APEX fused, FLAG fused, and endogenous CoIP; stable cell lines RNA sequencing; in vivo liver and brain RNA sequencing for 2 hours, 14 days, and 28 days; and ELISA targeted quantification in human tissues.

It is further conceived that in some embodiments, SHMOOSE or a fragment thereof or an SNP variant thereof is administered via intracerebroventricular injection. Since SHMOOSE attenuates weight gain on mice fed with a high fat diet and enters the circulatory system, it is likely that the peripheral effect of SHMOOSE is mediated through neurobiological mechanisms. It is also conceived that SHMOOSE can serve as a therapeutic target for dementia, given the associative effects of SHMOOSE with neurodegeneration and “drugability” of peptides. Therefore, in some embodiments, methods are conceived to treat or reduce severity of dementia by administering an agent that targets or specifically binds (or blocks) SHMOOSE, e.g., an antibody against SHMOOSE or a fragment thereof. This may be tested in a triple-transgenic mouse model of AD (3×Tg-AD) which expresses Aβ plaques and tau-laden neurofibrillary tangles, as well as synaptic and behavioral deficits.

### Example 10. Mitochondrial Rs2853499 Occurs within a Previously Unannotated Microprotein and Associates with AD and Neuroimaging Phenotypes

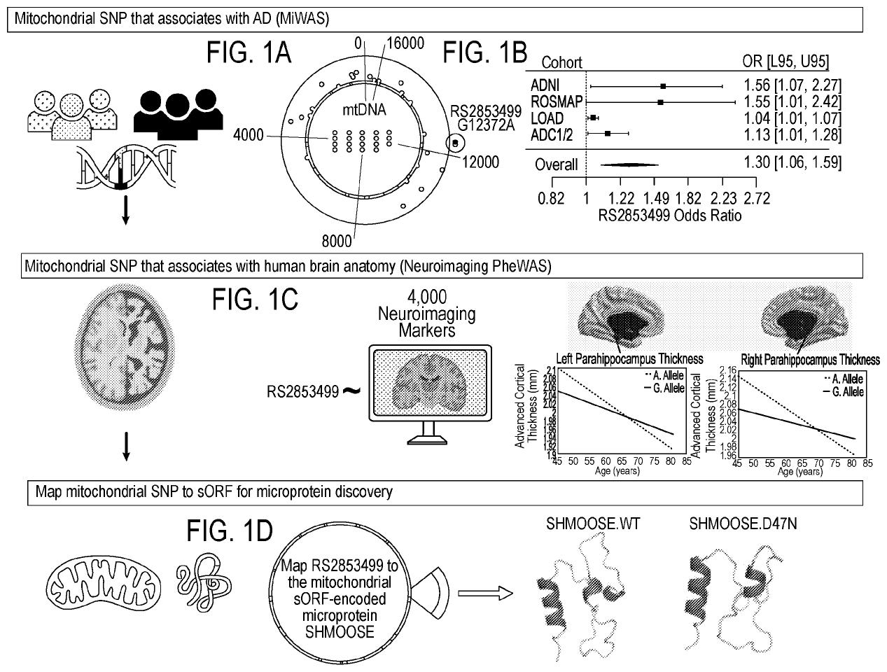

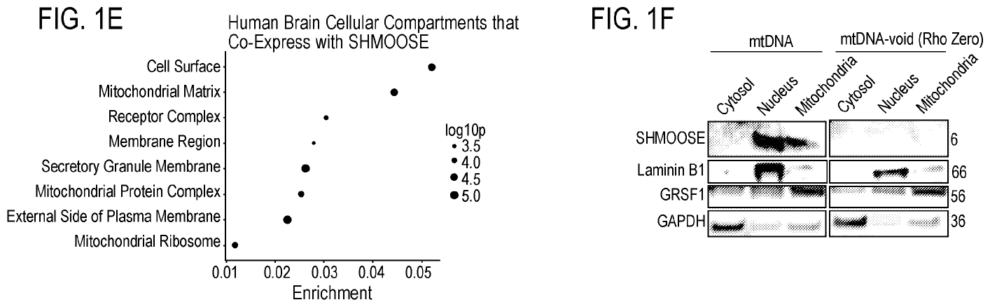

First, the inventors tested the hypothesis that mitochondrial SNPs within sORFs will associate with AD. Previously, it has been reported that mitochondrial haplogroup U associated with AD in the ADNI1 cohort (n=˜300). Since then, ADNI has expanded its cohort size with multiple new phases, which the inventors included in the mitochondrial-wide association study (MiWAS). By assessing ADNI 1, GO, 2, and 3 (n=˜800), the inventors confirmed haplogroup U SNPs (i.e., base pair positions 11467, 12308, and 12372) associated with AD (FIG. 1A). These three haplogroup SNPs do not change the amino acid sequences of the mt-ND4 and mt-ND5 proteins. However, mtSNP at base pair position 12372 (rs2853499) does change the amino acid sequence of a microprotein encoded by a sORF, SHMOOSE (Small Human Mitochondrial ORF Over SErine tRNA). Specifically, rs2853499 (henceforth referred to as SHMOOSE.D47N) changes the 47th amino acid from glutamine to aspartic acid (FIG. 1D). In ADNI, SHMOOSE.D47N carriers presented an odds ratio of 1.56 (case frequency: 22.9%; control frequency: 15.7%; 95% CI: 1.06-2.30; permutation 86 empirical p value<0.03).

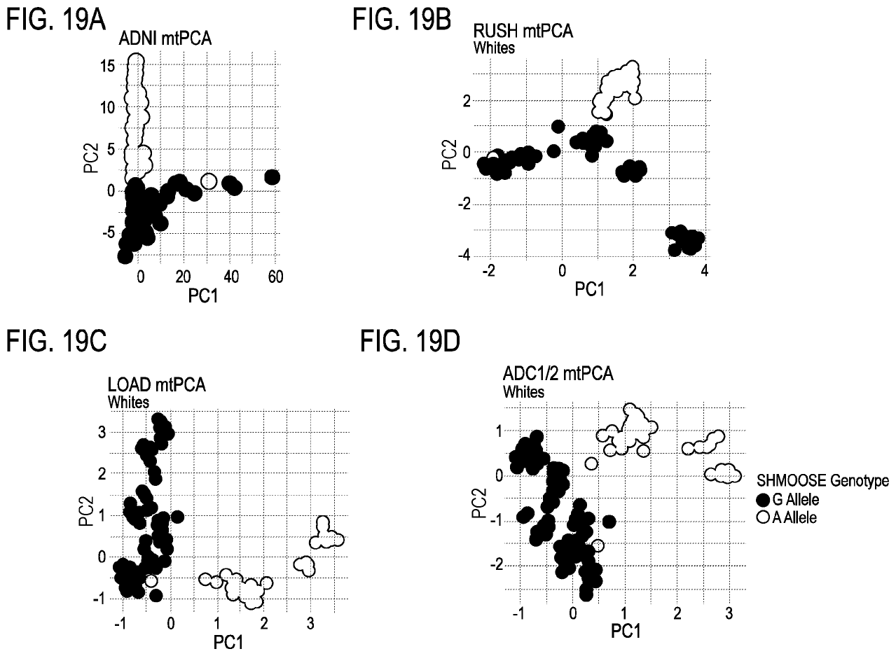

Furthermore, the inventors examined the effects of SHMOOSE.D47N in three additional cohorts: Religious Orders Study (ROS) and Memory and Aging Project (MAP), Late-Onset Alzheimer's Disease (LOAD), and NIA Alzheimer Disease Centers (ADC1 and ADC2). SHMOOSE.D47N carriers in ROSMAP, LOAD, and ADC1/2 presented odds ratios, respectively, of 1.55 (case frequency: 25.3%; control frequency: 17.6), 1.04 (case frequency: 24.7%; control frequency: 23.0%) and 1.13 (case frequency: 24.0%; control frequency: 23.3%). The inventors analyzed these cohorts with consideration to cohort-specific allele frequency differences. Whereas the inventors did not observe significant mitochondrial genetic heterogeneity for SHMOOSE.D47N carriers in ADNI and ROSMAP, the inventors did observe mitochondrial genetic heterogeneity for SHMOOSE.D47N in LOAD and ADC1/2, which the inventors corrected in statistical models (FIG. 19). Altogether, random effects meta-analysis estimated an odds ratio of 1.30 for SHMOOSE.D47N (95% CI: 1.06-1.59; p value<0.005; FIG. 1B). Also see Table 2.

TABLE 2
SHMOOSE mtSNP Effect on AD Across Cohorts

| | Case | | Control | | | p | | |
|---|---|---|---|---|---|---|---|---|
| Cohort | Frequency | Case n | Frequency | Control n | OR | value | L95 | U95 |
| ADNI | 22.7% | 480 | 15.7% | 321 | 1.56 | 0.022 | 1.07 | 2.27 |
| ROSMAP | 25.3% | 281 | 17.6% | 233 | 1.55 | 0.049 | 1.01 | 2.42 |
| LOAD | 24.7% | 993 | 23.0% | 883 | 1.04 | 0.016 | 1.01 | 1.07 |
| ADC½ | 23.4% | 2261 | 24.0% | 654 | 1.13 | 0.04 | 1.01 | 1.28 |

Model: AD~SHMOOSE_mtSNP + age + biological_sex + mtPC1 + mtPC2 + mtPC3
Note:
ADNI and ROSMAP not adjusted for mitochondrial PCs due to no SHMOOSE mtSNP admixture

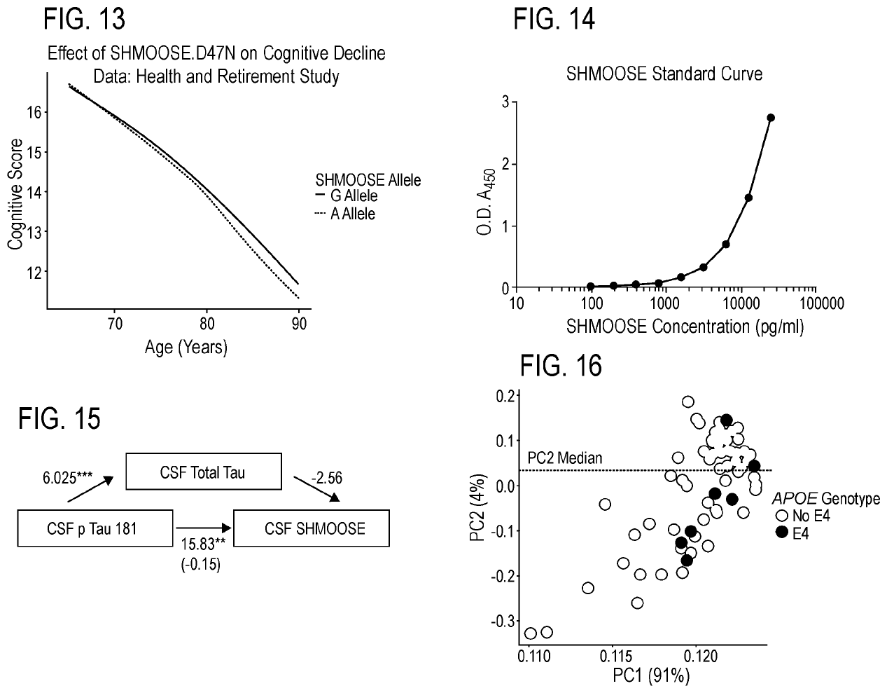

In addition, the inventors estimated the effect of SHMOOSE.D47N on cognitive decline in the Health and Retirement Study (HRS), a population-based study of US adults aged 50 years or older (n=˜15,000) 16, noting that alternative allele carriers had faster cognitive decline over time (FIG. 13). Since individuals with the SHMOOSE mtSNP (A allele) were predicted to have accelerated cognitive decline, these individuals can be identified as subjects for early treatment for cognitive decline.

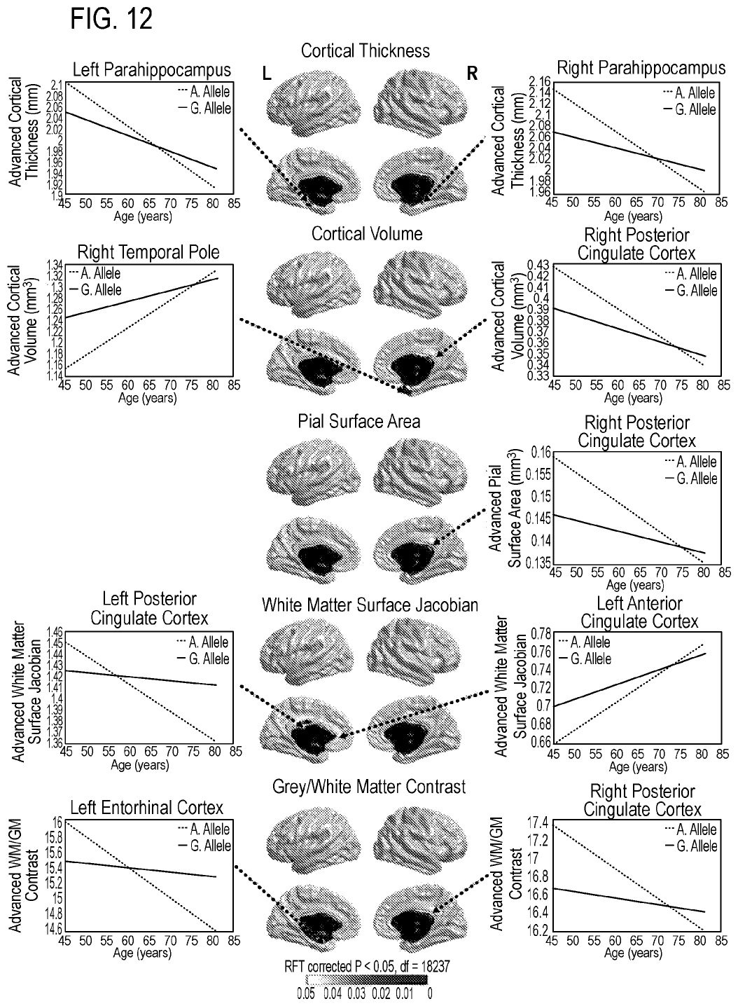

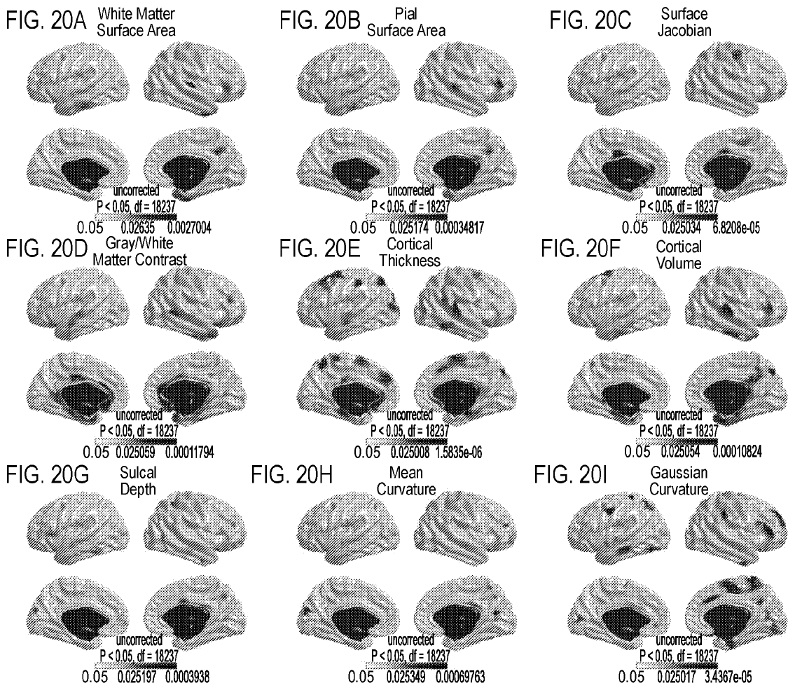

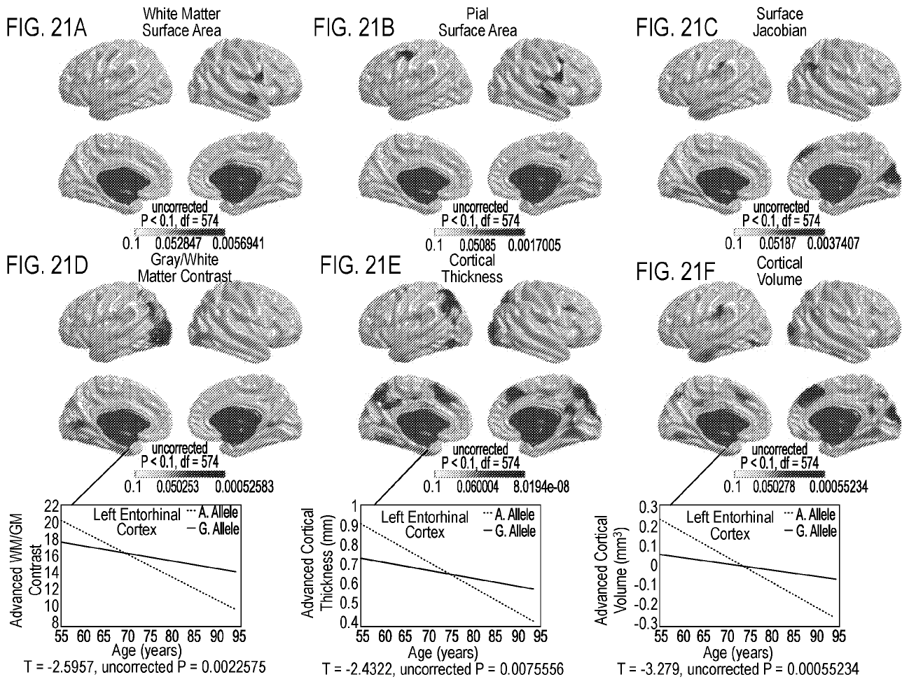

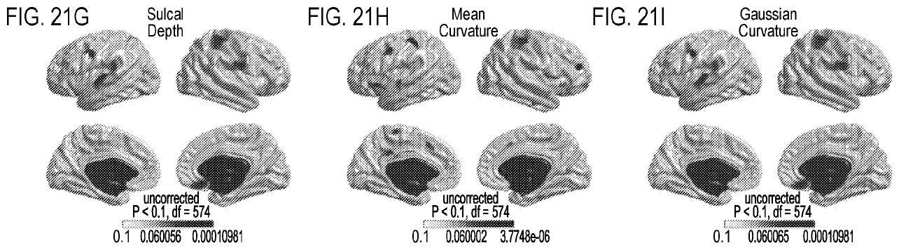

Since GWAS/MiWAS are prone to spurious associations, the inventors carried out a phenome wide association study (PheWAS) that included approximately 4,000 neuroimaging modalities on a large sample of ˜18,300 European-ancestral individuals. A significant advantage of PheWAS is the replication of SHMOOSE.D47N across related neurobiological phenotypes, which is both targeted and statistically robust due to abundant power. In PheWAS, SHMOOSE.D47N (frequency: 25.5%) significantly associated with cortical thickness, volume, pial surface area, WM surface Jacobian, and GM/WM contrast in several paralimbic regions, including the parahippocampal gyri, the entorhinal cortex (EC), the anterior cingulate cortex (ACC), the posterior cingulate cortex (PCC), and the temporal pole (TPO) (clusterwise, RFT-corrected p value<0.05; FIG. 12). Over age-span, SHMOOSE.D47N carriers exhibited accelerated thinning of the parahippocampus (FIG. 1C), EC, and PCC. That is, the crossed age trajectories of SHMOOSE.D47N showed inversed effects at younger and older ages. For instance, the SHMOOSE reference allele associated with smaller brain structural measures at middle (45-65 years) and/or young-old (65-75 years) ages, whereas the alternative allele (i.e., SHMOOSE.D47N) associated with structural loss at old ages (>75 years). In addition to the predominant results in the paralimbic areas, at a lenient threshold of uncorrected p value<0.05, the inventors observed distributed trends of SHMOOSE.D47N effects in the language centers (superior temporal and inferior frontal gyri), dorsolateral and medial prefrontal cortex, central motor, and occipital visual cortices (FIG. 20). Separately, the inventors observed similar trends in the ADNI PheWAS, which was conducted on the same sample used for ADNI permutation MiWAS. As the inventors observed in UK Biobank, SHMOOSE.D47N significantly associated with limbic regions such as the medial temporal cortex and posterior cingulate cortex at a lenient threshold of uncorrected p value<0.05 (FIG. 21).

Since rs2853499 associated with neuroimaging outcomes and AD—and that the variant mutates the SHMOOSE amino acid sequence (FIG. 1D) —the inventors sought to detect endogenous SHMOOSE. Therefore, the inventors started by assessing the 131 correlations between SHMOOSE RNA counts and nuclear-encoded gene counts on 69 non-demented post mortem brains temporal cortices. These analyses revealed the mitochondria cellular compartment co-expressed with SHMOOSE (FIG. 1E). Indeed, using a custom antibody against the c-terminus of SHMOOSE, the inventors detected SHMOOSE via Western blot from neuronal mitochondria and nuclei fractions at the predicted ˜6 kDa molecular weight (FIG. 1F). Yet in rho zero cells (i.e., cells without mtDNA), the inventors did not detect SHMOOSE, further confirming SHMOOSE is derived from mitochondrial DNA.

Altogether, the inventors targeted and detected the novel microprotein SHMOOSE after identifying a genetic variant within its sORF that associated with AD and brain structure.

### Example 11. SHMOOSE Levels in Cerebrospinal Fluid Correlate with Age, Tau, and Brain White Matter Microstructure

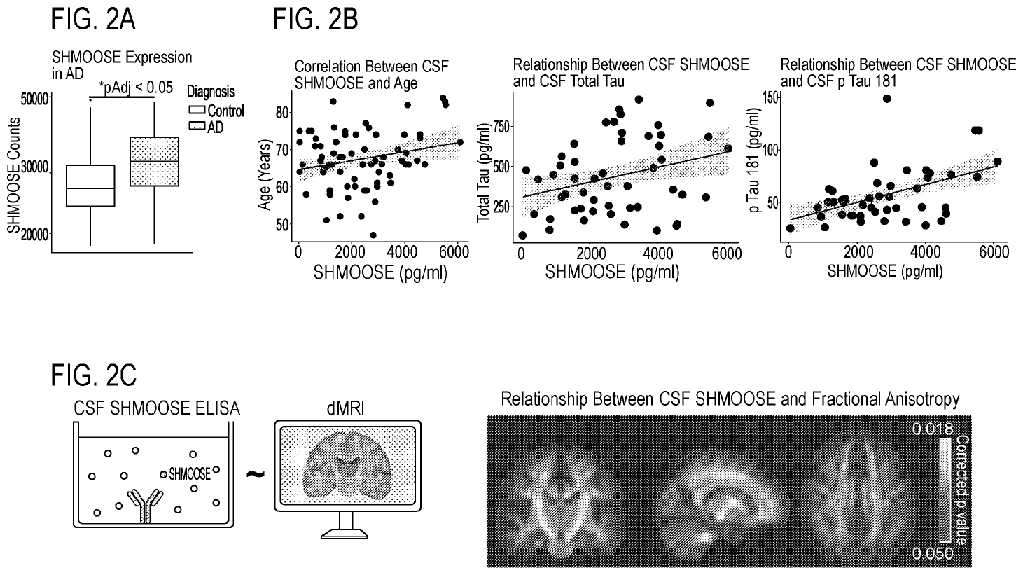

A new assay or test was developed to quantify levels of SHMOOSE in biological tissues. In particular, an enzyme linked immunosorbent sandwich assay (ELISA) was developed by using an antibody against amino acids 32-58 of SHMOOSE. SHMOOSE levels in CSF correlate to age, tau, and brain white matter microstructure. In the temporal cortex of AD brains, SHMOOSE RNA was ˜15% greater compared to controls (AD n=82; control n=78; FIG. 2A). That is, RNA levels of SHMOOSE are higher in the temporal cortex of Alzheimer's disease brains. But to assess microprotein levels directly, the inventors developed a SHMOOSE enzyme-linked immunosorbent assay (ELISA) with sensitivity ranging from 100-250,000 pg/ml (FIG. 14). In 79 cerebrospinal samples (CSF) of non-demented individuals from the University of Southern California (USC) AD Research Center, the inventors analyzed the effects of age, CSF amyloid beta, CSF total tau, CSF phosphorylated tau at residue number 181 (p tau 181), and white matter microstructure (measured by diffusion tensor imaging fractional anisotropy) on SHMOOSE levels. CSF SHMOOSE levels positively and significantly correlated with age, CSF total tau, and CSF p tau 181 (FIG. 2B). Thus, actual peptide levels of SHMOOSE in cerebrospinal fluid correlate with age, tau, and phosphorylated tau. To determine if the effect of p tau 181 on SHMOOSE levels was mediated through total tau, the inventors performed a mediation analysis in which total tau was considered the mediator. The indirect effect of this mediation was not considered statistically significant, and the direct effect of p tau 181 on SHMOOSE tau was slightly attenuated when controlling for total tau (p value=0.060). Together, the direct and indirect effect of p tau 181 on SHMOOSE was significant (p value<0.01). Therefore, p tau 181 appears to be specifically driving the relationship with SHMOOSE and is not mediated by total tau (FIG. 15). Separately, the inventors did not observe significant correlations between SHMOOSE and CSF levels of amyloid beta 42. Finally, the inventors found that individuals with higher CSF SHMOOSE levels also had lower DTI fractional anisotropy FA in the body of the corpus callosum and bilateral superior corona radiata (FIG. 2C), even after controlling for age, biological sex, and cognitive status (CDR test score).

### Example 12. SHMOOSE Associates with Mitochondrial and Ribosomal Gene Expression Changes

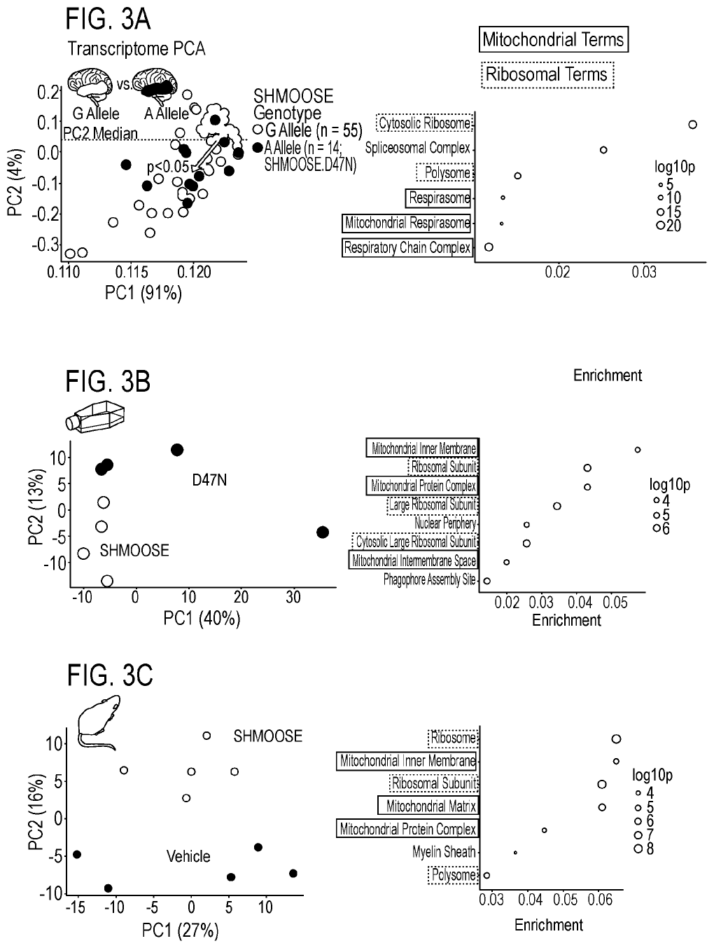

Given that the inventors observed neuroimaging modality differences by SHMOOSE genotype, the inventors further hypothesized brain gene expression will differ by SHMOOSE genotype (i.e., SHMOOSE.D47N). Therefore, the inventors analyzed RNA-Seq data derived from 69 post-mortem brain temporal cortexes (Mayo Clinic; n=14 alternative allele and n=55 reference allele). The inventors observed that SHMOOSE.D47N associated with 2,122 differentially expressed genes in the brain temporal cortex under a p adjusted value of 0.05. That is, gene expression differences were observed between SHMOOSE genotypes in Mayo Clinic Temporal Cortex RNA-Seq. Remarkably, principal components derived from the gene count matrix revealed clustering SHMOOSE reference allele gene expression, while the signature for SHMOOSE.D47N carriers drifted further from the reference allele cluster. By categorizing samples based on the median value of the second principal component, SHMOOSE.D47N carrier exhibited significant global gene expression deviation (p value<0.05; FIG. 3A). Considering that post-mortem brain gene expression is influenced by several factors (i.e., environment, etc.), the inventors consider the significant effects of SHMOOSE.D47N extremely noteworthy. As a contrast, the inventors noticed a similar trend for APOE4 carriers, in which APOE4 carriers trended away from the population norm, although the trend was not statistically significant (FIG. 16). These statistically differentially expressed genes by SHMOOSE.D47N carriers enriched GO cellular compartment terms for mitochondria and ribosomes.

Next, to determine whether the effects for SHMOOSE.D47N on gene expression can be recapitulated in vitro, the inventors conducted differential transcriptomics after administering SHMOOSE or SHMOOSE.D47N to neural cells for 24 hours. SHMOOSE.D47N-treated cells induced differential expression of 1,400 genes under a p adjusted value of 0.2. That is, gene expression differences were observed between cells treated with SHMOOSE or SHMOOSE.D47N. Indeed, these significant genes enriched mitochondrial and ribosome cellular compartments, like what was observed by SHMOOSE genotype in the 69 post-mortem human brains. “Mitochondrial inner membrane” was the top enriched GO cellular compartment term (FIG. 3B).

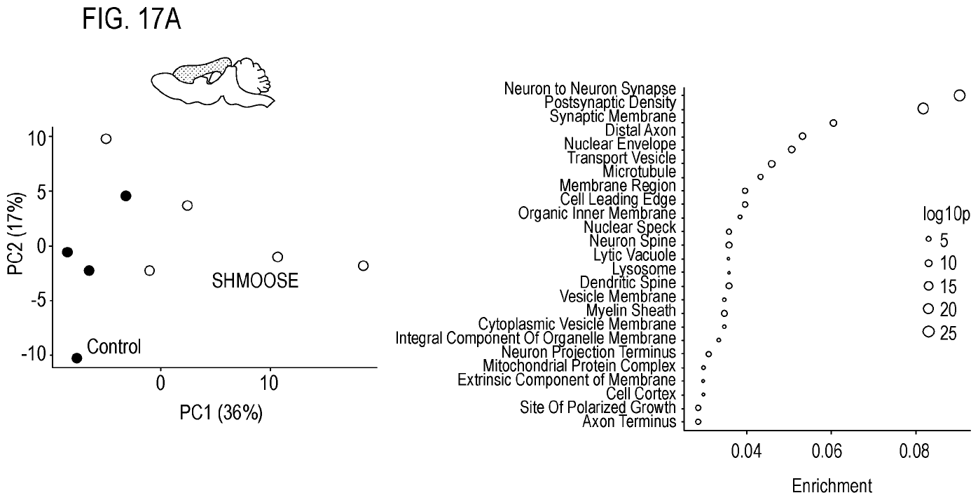

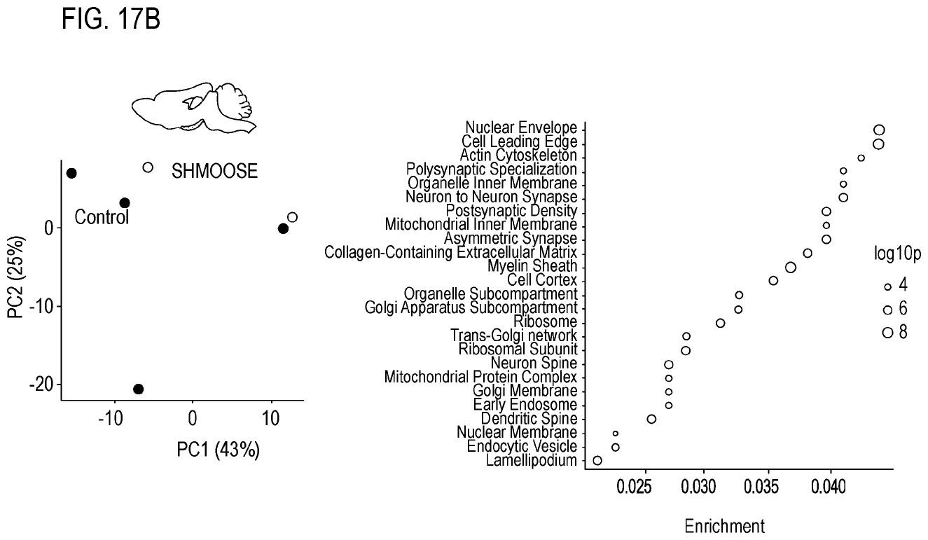

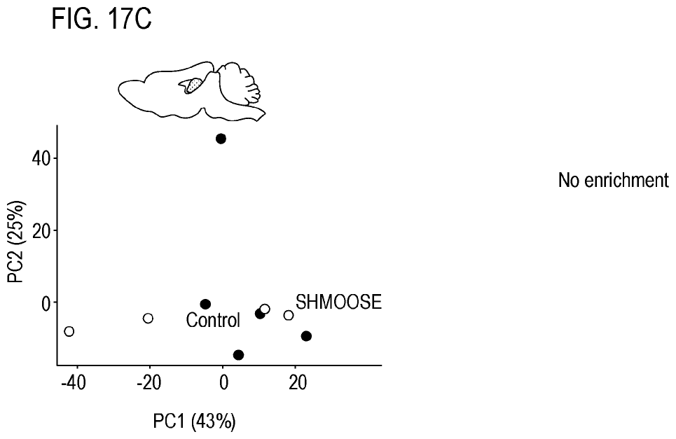

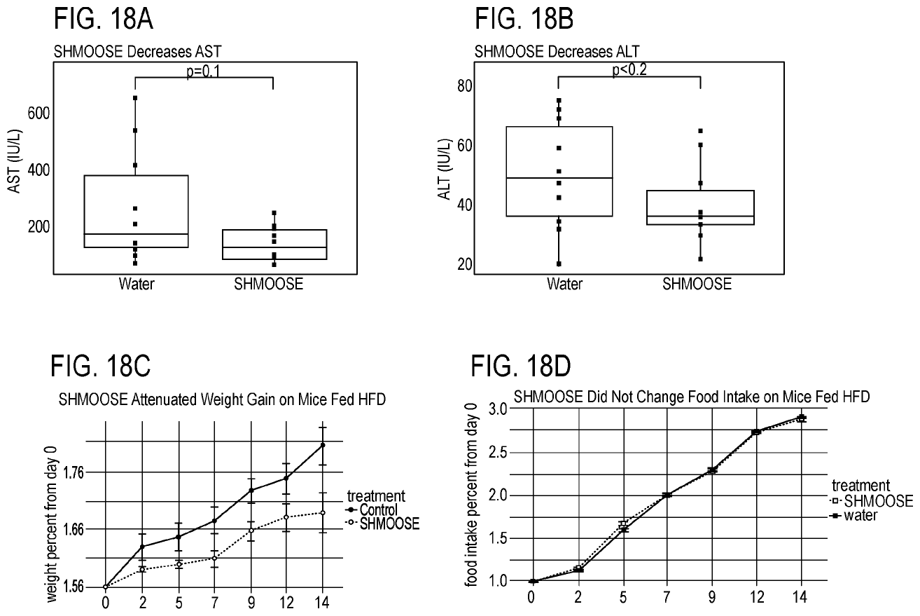

The inventors also injected SHMOOSE intraperitoneally (IP) to 12-week-old C57BL/6J mice fed a high fat diet (i.e., a mild metabolic perturbation). At the conclusion of the two-week study, the inventors harvested brain and liver for RNA-Seq. As the inventors observed for SHMOOSE.D47N carriers and in in vitro experiments, 367 differentially expressed genes under a p adjusted value of 0.05 enriched mitochondrial and ribosomal terms in the liver (FIG. 3C). That is, liver expression differences were observed after SHMOOSE IP injections. Brain hypothalamus expression differences were also observed after SHMOOSE IP injections. Further, cortical expression differences were observed after SHMOOSE IP injections. However, while the inventors observed global gene expression changes as illustrated by PCA in the mouse cortex and hypothalamus, most enriched terms included central nervous system-related terms, not ribosomes nor mitochondrial specifics (FIG. 17). Furthermore, brain hippocampus expression differences were observed after SHMOOSE IP injections. The inventors also did not observe robust gene expression differences in the hippocampus (FIG. 15). Mice treated with SHMOOSE did not display toxic effects nor behavior changes and showed attenuated weight gain and rise in the liver enzymes AST and ALT (without change in food intake), as compared to control treated mice (FIG. 18).

### Example 13. SHMOOSE Modifies Cellular Metabolic Activity and Protects Against Amyloid Beta Toxicity

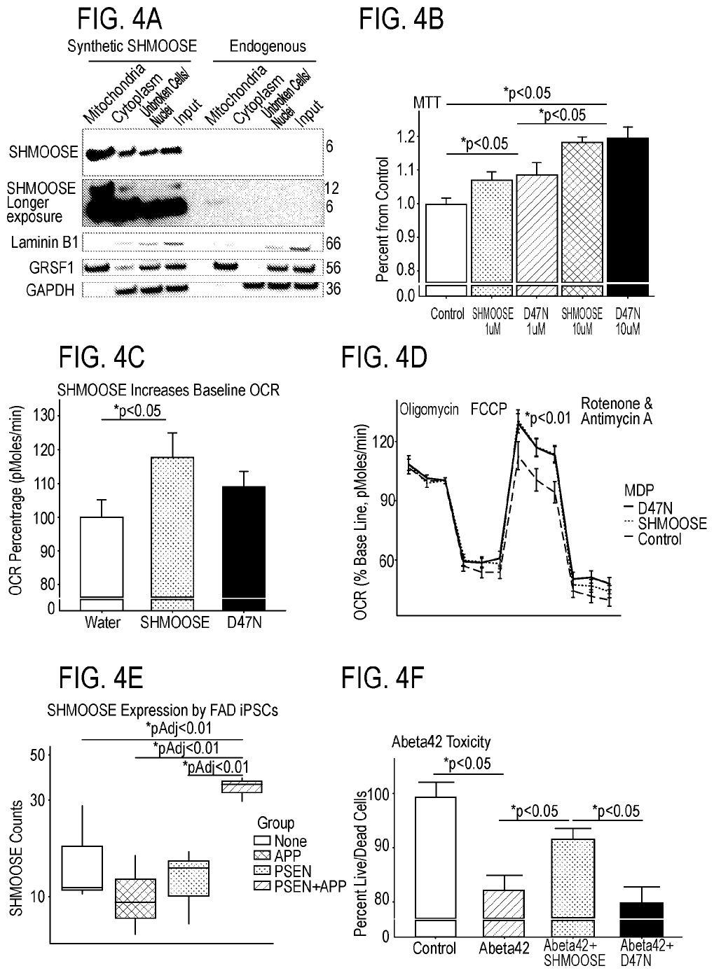

Since SHMOOSE associated with differential mitochondrial gene expression (FIGS. 4A-4C), the inventors hypothesized SHMOOSE would induce metabolic changes in vitro. Indeed, when SHMOOSE was exogenously administered to cells, the peptide localized prominently to mitochondria (FIG. 4A). In a dose-dependent response from 1 uM to 10 uM, both SHMOOSE and SHMOOSE.D47N increased neural cell metabolic activity by 10% and 20%, respectively (FIG. 4B). Thus, SHMOOSE, when given to cells, boosts metabolic activity in a dose response manner. During mitochondrial stress, in which maximum proton flow was permitted through the inner mitochondrial membrane following oligomycin and FCCP administration, both SHMOOSE and SHMOOSE.D47N boosted mitochondrial spare capacity (FIG. 4C), suggesting general effects for both forms of SHMOOSE on mitochondrial biology. However, compared to SHMOOSE.D47N, wild type SHMOOSE significantly increased basal oxygen consumption rate by approximately 20% (FIG. 4D).

Additionally, in neurons derived from iPSCs with both APP and PSEN1 mutations—two familial AD mutations—SHMOOSE RNA expression was three-fold higher than neurons derived with just one mutation, suggesting a role for SHMOOSE in amyloid beta biology (FIG. 4E). Likewise, in neuronal cells stressed with oligomerized amyloid beta, SHMOOSE administration protected against cell death, but SHMOOSE.D47N did not similarly protect cells (FIG. 4F).

### Example 14. SHMOOSE Interacts with the Inner Mitochondrial Membrane Mitofilin

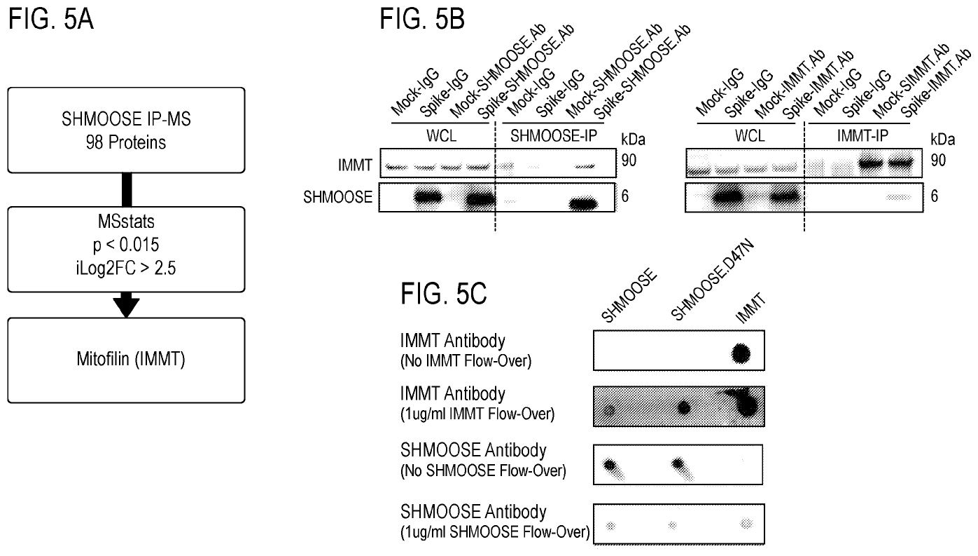

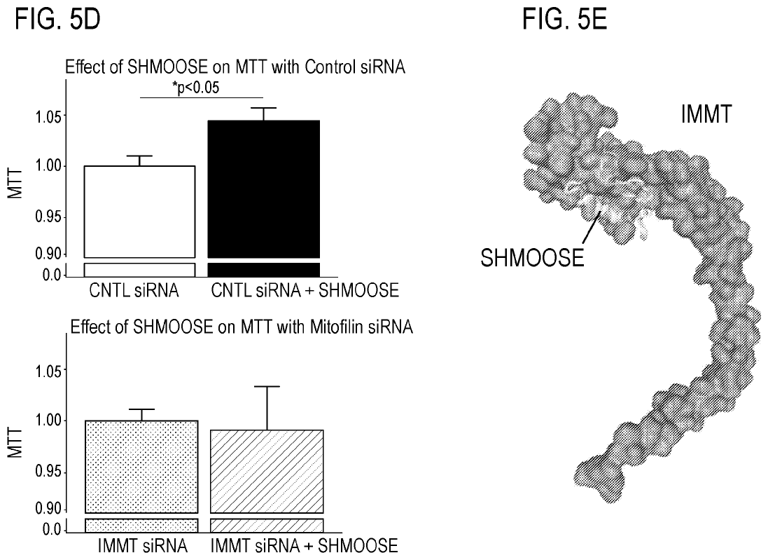

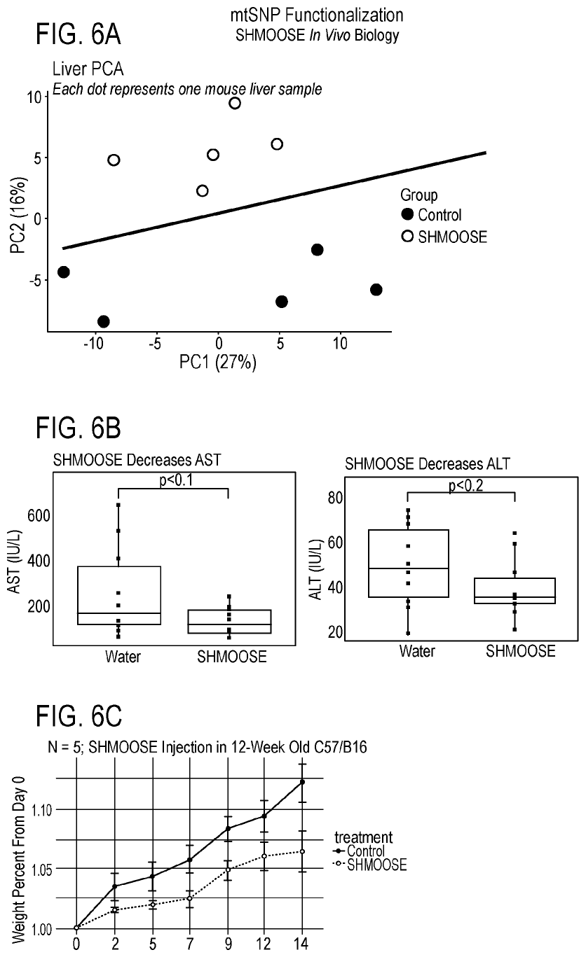

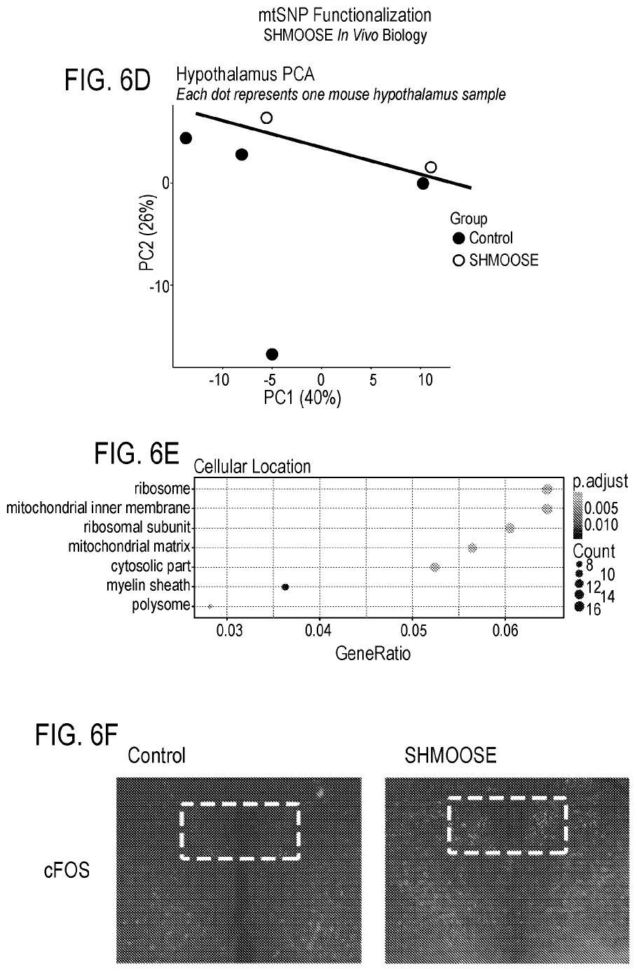

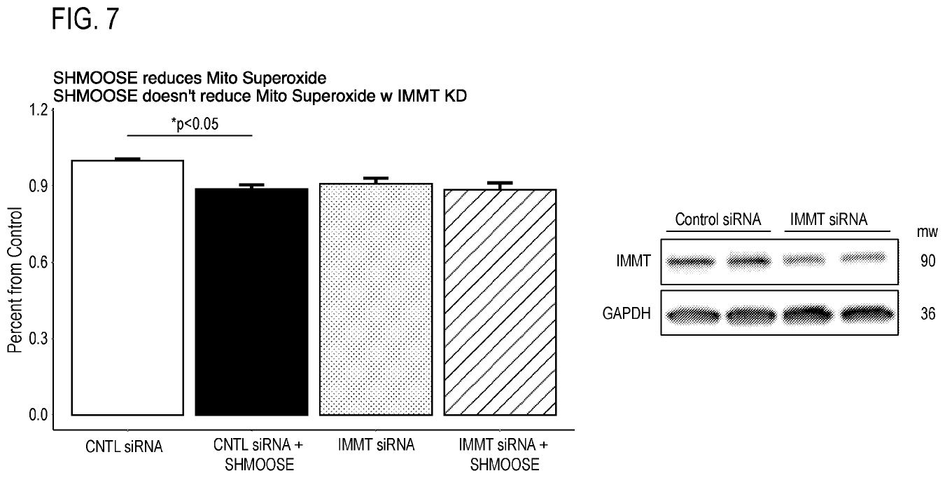

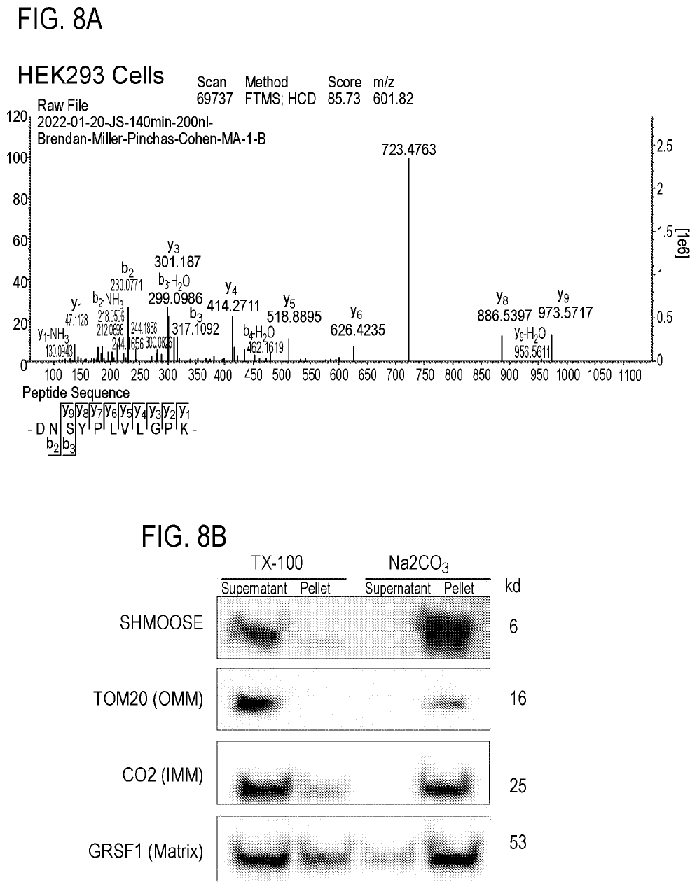

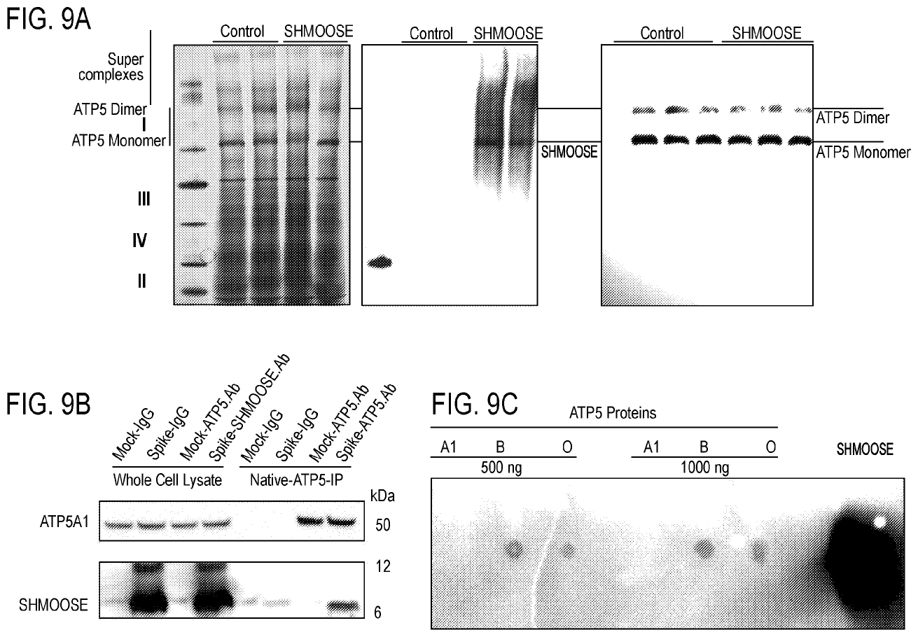

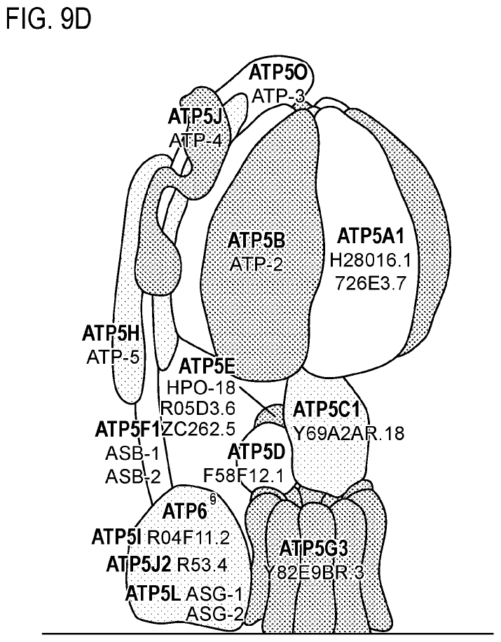

The inventors searched for SHMOOSE protein interacting partners by conducting co-immunoprecipitation assays. Ultimately, the inventors identified mitofilin as a protein interacting partner with SHMOOSE. Mitofilin was targeted based on proteomics analysis of neuronal lysates that were spiked with SHMOOSE followed by SHMOOSE antibody-based immunoprecipitation. Mass spectrometry-based analysis of these lysates suggested SHMOOSE bound 98 proteins; however, during protein quantification filtering and indexing by p value and fold change, the inventors considered mitofilin a top SHMOOSE binding protein candidate (FIG. 5A). Indeed, after administering SHMOOSE to neural cells, the inventors validated the SHMOOSE-mitofilin interaction by reciprocal immunoprecipitation followed by Western blot (FIG. 5B). In addition, the inventors conducted reciprocal dot-based immunoblots between recombinant SHMOOSE and mitofilin, confirming binding between mitofilin and SHMOOSE as well as SHMOOSE.D47N (FIG. 5C). Moreover, after knocking down mitofilin using siRNA, the inventors observed no effect of SHMOOSE on neural cell metabolic activity, as measured by MTT assay (FIG. 5D). The inventors modeled the biophysical interaction between SHMOOSE and mitofilin using HDOCK, a hybrid algorithm of template-based and template-free docking (FIG. 5E), noting that the predicted interacting between SHMOOSE and mitofilin center on the c-terminus of mitofilin (residues 332-413).

### Discussion

The inventors initially targeted SHMOOSE because a mitochondrial SNP within the SHMOOSE sORF associated with AD, neuroimaging modalities, and brain gene expression in large epidemiological cohorts. In four cohorts, individuals with SHMOOSE.D47N exhibited increased risk for AD (OR: 1.30; FIG. 1B). In neuroimaging based PheWAS, SHMOOSE.D47N carriers had greater atrophy over age in medial temporal areas such as the parahippocampus, entorhinal cortex, and anterior and parietal cingulate cortex. The medial temporal cortex and parietal cingulate cortex are known to be vulnerable in Alzheimer's disease (AD), and the pathological atrophy likely appears years before clinical symptoms. The inventors also examined brain neuroimaging in ADNI and observed similar effects in the limbic area, but no results survived multiple correction, and the inventors lacked the power that UK Biobank (n=˜18,300) provided. Moreover, the crossed age trajectories of SHMOOSE.D47N showed inversed effects at younger and older ages. The SHMOOSE reference allele associated with smaller brain structural measures at middle (45-65 years) and young-old (65-75 years) ages, whereas the alternative allele was associated with structural loss at old ages (>75 years). Such contrary genetic effects in younger and older samples have been observed for several other genes that were frequently associated with age-sensitive cognitive functions and neurodegeneration (e.g., brain-derived neurotrophic factor).

Given the genetic association between this SHMOOSE SNP and neurobiological phenotypes (i.e., AD and neuroimaging modalities), the inventors targeted SHMOOSE biochemically by developing a polyclonal antibody against amino acid residues 32-58 of SHMOOSE. Likewise, the inventors detected SHMOOSE at the predicted ˜6 kDa via Western blot in neuronal mitochondrial as well as nuclei, while the inventors did not observe SHMOOSE detection in cells void of mitochondrial DNA. Furthermore, the inventors found CSF SHMOOSE positively correlated with age, tau, and brain white matter. Since higher levels of CSF tau have previously predicted AD, correlations between SHMOOSE and tau suggest SHMOOSE could be involved in the progressive etiology of AD and can be a biomarker. Moreover, the inventors observed that higher CSF SHMOOSE levels associated with DTI FA in non-demented older adults. Various factors can contribute to lower DTI FA, but it may reflect lower levels of myelination and is often associated with a disease state. Myelin maintenance and repair is metabolically demanding and is particularly vulnerable to damage when energy deficits exist, providing a possible link between a mitochondrial peptide and white matter microstructure. Possibly the relationship between higher CSF SHMOOSE and lower regional DTI FA indicates an incomplete compensation for metabolic or other stressors in the brain.

The inventors further assessed the SHMOOSE SNP (i.e., SHMOOSE.D47N) in human population cohorts to infer biological mechanism. Remarkably, the SHMOOSE.D47N SNP alone differentiated the human brain transcriptome, as the gene expression signature via PCA of post-mortem brains with SHMOOSE.D47N drifted from SHMOOSE reference allele cluster. This was surprising given the reported effects of environment, lifespan, etc. on human brain transcriptomics. Likewise, in vitro, gene expression differences by SHMOOSE and SHMOOSE.D47N enriched inner mitochondrial membrane and ribosomes compartments. Furthermore, in in vivo studies involving IP injections of SHMOOSE, the inventors also observed ribosomal and mitochondrial inner membrane gene expression changes beyond the brain in the liver, suggesting that SHMOOSE could act on non-neural systems. These mice treated with SHMOOSE experienced attenuated weight gain during high-fat diet ad lib with mild reductions in liver enzymes ALT and AST.

In all transcriptomics studies, the inventors observed a common theme for mitochondrial inner membrane enrichment, which the inventors consider noteworthy because SHMOOSE bound the inner mitochondrial membrane mitofilin in multiple models. Mitofilin is a component of the MICOS complex that regulates mitochondrial crista junctions and inner membrane organization. Separately, the inventors observed transcription enrichment for ribosomal terms, which might be explained through SHMOOSE-mitofilin interaction, as the Pathway Commons Protein-Protein Interactions data set shows nearly 1800 interacting proteins to mitofilin, 137 of which are ribosomal proteins.

Although past MiWAS studies have examined the effects of mtSNPs on neurodegeneration, few have followed up experimentally. One functional limitation of MiWAS is isolating the effects of individuals mtSNPs because these SNPs define broader haplogroups. As a result, the inventors cannot rule out that other SNPs within the SHMOOSE.D47N haplogroup (i.e., Haplogroup U) has effects independent from SHMOOSE (e.g., effects on tRNA). Nevertheless, the only missense effect of this SNP is to the SHMOOSE microprotein, and the inventors used this multi-phenotype strategy to identify a microprotein candidate for experimental validation (i.e., SHMOOSE).

The inventors' data has several implications. First, separate from the discovery of SHMOOSE, the inventors showed mitochondrial DNA variants can associate with several neurobiological phenotypes that can aid functional interpretation (i.e., disease classification, structural anatomy, and gene expression). That is, the inventors showed a naturally occurring version of SHMOOSE caused by a SNP associates with AD, brain gene expression, and brain anatomy in humans. Second, the inventors revealed mitochondrial DNA variants can be mapped to sORFs that encode biologically functional microproteins. As large human cohorts with genetic data continue to add whole genome sequencing data, it is foreseeable that this refined mtDNA resolution will yield additional microproteins. Moreover, as proteomics technology improves, it is also conceivable more mitochondrial encoded microproteins will be detected. Third, the correlation among CSF levels of SHMOOSE, CSF AD-related biomarkers (e.g., tau), and brain white matter suggests SHMOOSE has potential as a biomarker. Finally, SHMOOSE appears to be another microprotein that affects mitochondrial biology, as recent microprotein discoveries (e.g., mitoregulin, BRAWNIN, MIEF-MP1) have also noted profound effects on mitochondrial biology.

### Methods

Mitochondrial-Wide Association Study (MiWAS) and mtSNP AD Association Analyses

Effects of mitochondrial genetic variants on probable AD in ADNI1, ADNI GO, ADNI2, and ADNI3 were tested. The inventors followed up on MiWAS results that were previously reported on ADNI1. In the inventors' analyses, mitochondrial genotypes and diagnosis from ADNI1, ADNI GO, ADNI2, and ADNI3 were all merged for analysis. ADNI1 samples were genotyped using the Illumina 610-Quad BeadChip, and ADNI GO/2 samples were genotyped using the Illumina HumanOmniExpress BeadChip, which does not contain mtSNPs. Nevertheless, ADNI1/GO/2 samples were whole genome sequenced and include mitochondrial genotypes. These whole mitochondrial genotypes were processed with stringent quality controls and made available in variant call format. ADNI3 samples were genotyped using the Illumina Infinium Global Screening Array v2 (GSA2). Mitochondrial whole genome sequencing data was converted to suitable format using PLINK (v1.9) and merged with ADNI1 and ADNI3 in PLINK bed/bam/bim format. After merging genetic data, 138 mtSNPs remained for a total of 448 clinical probable cases and 290 controls during MiWAS. The MiWAS permutation model included a minor allele frequency threshold of 5% on individuals of European descent noted by ADNI, leaving 29 mtSNPs qualified for permutation. The inventors further assessed the degree of mitochondrial genetic admixture by conducting a principal component analysis on mtSNPs. These principal components were generated via singular value-decomposition of the mtSNP matrix, outputting eigenvectors that approximates the matrix with a minimal number of values (prcomp function in R), as portrayed elsewhere. The degree of mitochondrial genetic admixture was low and ideal for a permutation approach. Any mtSNP with an empirical p value under 0.05 was considered statistically significant. A total of 957 permutations were conducted for the most significant mtSNP, which occurred in the SHMOOSE sORF and became microprotein candidate. Separately, the inventors estimated the effect of the SHMOOSE mtSNP in the Rush Alzheimer's Disease Center (RADC) comprised of Religious Orders Study (ROS) and Memory and Aging Project (MAP), Late-Onset Alzheimer's Disease (NIA-LOAD), and NIA Alzheimer Disease Centers (ADC1 and ADC2) cohorts using logistic regression. ROSMAP samples were genotyped using whole genome sequencing, and mitochondrial genetic variants were made available to qualified users in VCF format. LOAD samples were genotyped using the Illumina 610-Quad BeadChip, and the ADC1 and ADC2 samples were genotyped using the Illumina Human660W-Quad BeadChip. Mitochondrial genetic admixture in ROSMAP (n=281 cases and n=233 controls), LOAD (n=993 cases and 374 n 883 controls) and ADC1/2 (n=2261 cases and n=654 controls) were also assessed by implementing mitochondrial principal component analysis (FIG. 13); the inventors observed mitochondrial genetic heterogeneity for SHMOOSE alternative allele carriers in LOAD and ADC1/2 and therefore included the first three mitochondrial principal components in the logistic regression model, with age and biological sex as additional covariates. For meta-analysis, age and biological sex were included in the ADNI model. After the effects of the SHMOOSE mtSNP were estimated in ROSMAP, LOAD, and ADC1/2, meta-analysis was conducted using a random effects model approach from the metafor R package.

### Neuroimaging Phenome-Wide Association Study (PheWAS)

This work was conducted using ADNI and the UK Biobank Resource (ukbiobank.ac.uk) under approved project 25641. The inventors used brain MRI imaging (UK Biobank data-field: 110) from the 2018 August release of 22,392 participants (biobank.ctsu.ox.ac.uk/crystal/label.cgi?id=110). Details of the MRI acquisition is described in the UK Biobank Brain Imaging Documentation (biobank.ctsu.ox.ac.uk/crystal/refer.cgi?id=1977) and in a protocol form (biobank.ctsu.ox.ac.uk/crystal/refer.cgi?id=2367). This study discarded 1,002 participants whose MRI scans did not pass manual quality assessment, 45 participants due to data withdrawal or failed image processing, and 3,055 participants who did not have white British ancestry and/or did not pass the sample quality control for the genetic data (biobank.ctsu.ox.ac.uk/crystal/label.cgi?id=100313), resulting a sample of 18,330 individuals with age range from 45 to 81 years (mean age=63.27+7.45 years), 8,729 males (47.62%), and 4,680 SHMOOSE mtSNP carriers (25.53%). All MR images were processed using the FreeSurfer software package v6.0 (surfer.nmr.mgh.harvard.edu) to extract brain-wide morphological measures. The FreeSurfer workflow includes motion correction and averaging of volumetric T1-weighted images, removal of non-brain tissue, automated Talairach transformation, brain volume segmentation, intensity normalization, tessellation of the boundary between gray matter (GM) and white matter (WM), automated topology correction, and surface deformation following intensity gradients to optimally place the GM/WM and GM/cerebrospinal fluid borders at the location where the greatest shift in intensity defines the transition to the other tissue class. Each hemispheric GM and WM surface is composed of 163,842 vertices arranged as 327,680 triangles. Once the surface models are complete, a number of deformable procedures were performed for further data processing and analysis, including surface inflation, registration to a spherical atlas using individual cortical folding patterns to match cortical geometry across subjects, and finally creation of a variety of surface-based brain morphological metrics. All the procedure for MRI processing were implemented on the LONI pipeline system (pipeline.loni.usc.edu) for high-performance parallel computing. This study included 9 vertex-wise brain morphological measures: cortical thickness, volume, WM surface area, pial surface area, sulcal depth, WM surface Jacobian, GM/WM contrast, mean curvature and Gaussian curvature. Detailed information about these surface-based metrics is available at surfer.nmr.mgh.harvard.edu/fswiki/. Briefly, cortical thickness values were calculated as the shortest distance between the gray and white matter surfaces at each vertex. Vertex-wise volume is calculated by dividing each obliquely truncated trilateral pyramid between the GM and WM surfaces into three tetrahedra. Vertex-wise surface area measures on the pial and WM surfaces are estimated by assigning one third of the area of each triangle to each vertex. Sulcal depth conveys information on how far removed a particular vertex point on a surface is from a hypothetical mid-surface that exists between the gyri and sulci. It gives an indication of linear distance and displacements: how deep and high are brain folds. Surface Jacobian measures how much the surface is distorted to register to the spherical atlas. GM/WM contrast presents the vertex-by-vertex percent contrast between white and gray matter, where WM is sampled 1 mm below the white surface, and GM is sampled 30% the thickness into the cortex. Mean curvature is the average of the two principal curvatures at a vertex. The Gaussian curvature is the product of the two principal curvatures at a vertex. Prior to statistical analysis, these surface-based data were smoothed on the tessellated surfaces using a Gaussian kernel with the full width half maximum of 20 mm to increase the signal-to-noise ratio and to reduce the impact of mis-registration. All UK Biobank participants were genotyped using the Affymetrix UK BiLEVE Axiom array (on an initial ˜50,000 participants) and the Affymetrix UK Biobank Axiom array (on the remaining ˜450,000 participants) were genotyped using the Affymetrix UK Biobank Axiom array. SHMOOSE genotype was extracted from the genotyping array using the PLINK2.0 software. To capture population structure, the UKB team computed the top 40 principal components (PCs) from the high-quality genotyping dataset 53. Furthermore, to capture the population structure hidden in the mitochondrial genome, the inventors also computed mitochondrial PCs using a mitochondrial principal component analysis. To test effects of the SHMOOSE mtSNP on age-related brain structural differences, the inventors assessed the interaction between the SHMOOSE mtSNP genotype and age by implementing linear mixed-effects regression at each cortical surface vertex i for a given morphological measure Yᵢ with the model:

$${Y}_{i} = intercept + {β}_{1} G + {β}_{2} A g e + {β}_{3} A g e \times G + e i ,$$

where G is the SHMOOSE mtSNP genotype, Age is individuals' age in the scanner, e is the residual error, and the intercept and β terms are the fixed effects. Sex, intracranial volume (ICV), nuclear and mitochondrial PCs were added to the model as confounding variables. Statistical results at all vertices were corrected for the family-wise error rate (FWER) across the brain surface using the random field theory (RFT) method that adapts to spatial smoothness of the neuroimaging data. All surface-based analyses were conducted using Neuroimaging PheWAS system, which is a cloud-computing platform for big-data, brainwide imaging association studies.

### Cognitive Decline Analysis

In HRS, the inventors assessed the effect of SHMOOSE genotype on longitudinal cognitive decline over the aging process by implementing a mixed effects regression approach. HRS used the HumanOmni2.5 array to directly genotype 256 mitochondrial SNPs. SHMOOSE genotype was extracted using PLINK2.0. The validated HRS cognitive score represents episodic memory learning, episodic memory retrieval, semantic fluency, and orientation. The mixed effects model included fixed effect terms for biological sex, linear and quadratic age, mitochondrial genetic ancestry, and SHMOOSE genotype for European-ancestral individuals. Subject-specific random effects contained between individual variation at the age of 65 in addition to inter-individual variation in the rate of cognitive score change during aging (i.e., follow-up visits every two years). The lme4 package in R was used to carry out the analysis. A total of 8,072 individuals were individually assessed with 45,465 total data points.

### SHMOOSE Protein Structure Prediction

RoseTTAFold was used to predict the microprotein structure of SHMOOSE. Full algorithm details have been comprehensively detailed elsewhere. In comparison to Alphafold2, RoseTTAFold achieved similar degree of accuracy for complex proteins. The wild-type version of SHMOOSE and SHMOOSE.D47N were modeled, and output files were 472 downloaded into PDB format.

### Co-Expression Analysis

The inventor utilized transcriptome data generated by Mayo (Synapse ID: syn5550404). SHMOOSE transcript count matrices were created from made-available bam files. This was done by constructing a sORF database in GTF format and implementing the summarize Overlaps function of the GenomicAlignments package in R. Thereafter, normalized counts were used to conduct correlation between SHMOOSE counts and all nuclear-encoded gene counts, corrected for multiple hypotheses using a false discovery rate (FDR) of 0.05. Genes that statistically correlated with SHMOOSE expression were tested for enrichment using the enrichGo function from the clusterProfiler package, which returns enrichment of Gene Ontology (GO) categories after FDR control. Data output from the enrichGo function were used to generate plots using ggplot2 in R.

### Cells

SH-SY5Y cells used in the study were purchased from ATCC (CRL-2266). Cells were grown in DMEM/F12 with 10% FBS at 37° C. with 5% CO2 and split every 4-7 days depending on confluency. In addition, for rho zero cells, SH-SY5Y cells were depleted of mitochondrial DNA by adding 5 ug/ml ethidium bromide, 50 ug/ml uridine, and 1 mM pyruvate for approximately two months, as previously described. For all experiments, cells were differentiated by addition of 10 uM retinoic acid in DMEM/F12 with 1% FBS, and the media was changed once every 48 hours for a total of two changes, as described previously. When indicated, cells were treated with chemically synthesized SHMOOSE, which was made by GenScript by solid-phase peptide synthesis methods.

Trifluoracetic acid (TFA) was used to cleave synthesized peptide from resin. After peptide synthesis, residual TFA was removed and the pH of reconstituted SHMOOSE was neutral.

### Subcellular Fractionation

Cytosolic, nuclear, and mitochondrial fractions were prepared from cultured SH-SY5Y cells. To extract nuclei, cells were washed in ice-cold DPBS and resuspended in fractionation buffer containing 10 mM HEPES pH 7.6, 3 mM MgCl2, 10 mM KCl, 5% (v/v) glycerol, 1% Triton-X100, and protease/phosphatase inhibitors for 15 minutes, followed by centrifugation for 5 minutes at 250×g and 4° C. The resulting supernatant was further centrifuged once more at 18,000×g for 10 minutes at 4° C. to obtain a relatively pure cytoplasmic fraction. The original pellet prior to the 18,000×g centrifugation was washed in 10 mM HEPES pH 7.6, 1.5 mM MgCl2, 10 mM KCl, and protease/phosphatase inhibitors and centrifuged at 250×g and 4° C. The washed pellet was then resuspended in nuclear extraction buffer containing 20 mM HEPES pH 7.6, 1.5 mM MgCl2, 420 mM NaCl, 25% (v/v) glycerol, 0.2 mM EDTA, and protease/phosphatase inhibitors, followed by three sonication periods of 5 seconds (separated by 10 seconds) with 30% amplitude on ice. The sonicated pellet was centrifuged at 18,000×g for 10 minutes at 4° C. to obtain a relatively pure nuclear lysate. To extract mitochondria, cells were washed in ice-cold DPBS and resuspended in 2 ml hypotonic buffer containing 10 mM NaCl, 1.5 mM MgCl2, and 10 mM Tris-HCl pH 7.5 for 7.5 minutes. After the hypotonic incubation, cells were transferred to a glass homogenizer and homogenized by pressing straight down with the pestle 20 times, bursting the cells open while maintaining the integrity 516 of mitochondria. Mitochondrial homogenization buffer (MHB) was then added to the 2 ml homogenized sample to achieve a 1× concertation (210 mM mannitol, 70 mM sucrose, 20 mM HEPES, and 2 mM EGTA). The homogenate was then transferred to a clean 5 ml tube and centrifuged at 17,000×g for 15 minutes at 4° C. The resulting pellet was washed in MHB buffer and centrifuged two more times, followed by a resuspension of the mitochondrial pellet in RIPA lysis buffer, and final centrifugation step of 14,000×g for 10 minutes at 4° C. to obtain a relatively pure mitochondrial lysate. For exogenous SHMOOSE administration, 1 uM of SHMOOSE was administered to cells for 30 minutes, washed twice in cold PBS, and fractionated. 5-15 mg of protein were reduced in NuPAGE sample buffer and run on NuPAGE 4-12% Bis-Tris gels. Proteins were transferred to PVDF membranes, blocked with 5% BSA in TBS 0.1% tween, and incubated with respective antibodies at 1:1000 dilutions overnight at 4° C. The next day, membranes were washed with TBST 0.1% and incubated with 1:30,000 secondary antibody conjugated to HRP against the respective primary antibody species of origin, then excited using ECl reagent for 5 minutes.

### SHMOOSE Antibody Production and ELISA Development

Rabbit anti-SHMOOSE sera were produced by Yenzyme Antibodies (San Francisco, CA). SHOOSE affinity antibody was purified from rabbit anti-SHMOOSE sera using CarboxyLink Immobilization kit with UltraLink Support (Thermo Scientific) according to manufacturer's protocol. Briefly, anti-sera were applied onto the synthetic SHMOOSE peptide immobilized column and the eluted fractions were quantitated by UV absorbance at 280 nM. Circulating levels of SHOOSE were measured by in-house ELISA. Prior to assay, CSF was extracted with 90% acetonitrile and 10% 1N HCl. To measure endogenous SHMOOSE levels, synthetic SHMOOSE peptide was used as standard within range 100 pg/ml to 20,000 pg/ml. Briefly, 96-well microtiter plate was coated with anti-SHMOOSE polyclonal antibody for 3 hours followed by blocking the plate with SuperBlock buffer (Thermo Scientific). Next, standards, controls or extracted samples and pre-tittered detection antibody were added to the appropriate wells and incubated overnight. Followed by 3 washes, wells were added streptavidin-HRP conjugate and incubated for 30 minutes. After four washes, ultra-sensitive TMB (Thermo Scientific) were added and incubated for 10-20 minutes. The reaction was stopped by the addition of 2N sulfuric acid and absorbance was measured on a plate spectrophotometer at 450 nm. The intra- and inter-assay coefficient variations (CV) of SHOOSE ELISA were less than 10%, respectively.

### Correlation Between CSF SHMOOSE Levels and CSF Tau, CSF p Tau 181, and Brain DTI

The inventors considered data for 79 subjects recruited through the University of Southern California Alzheimer Disease Research Center (ADRC) who had available diffusion MRI (dMRI) scans and CSF measures of SHMOOSE. Of those, one did not have a usable dMRI scan and 6 were excluded for preprocessing failures identified through quality assessment (see Diffusion MRI Preprocessing). The final sample included 72 non-demented older (mean 65.7; 47-82 years old) adults who had diffusion MRI scans that passed all quality checks and available CSF measures of SHMOOSE. Subjects had a clinical dementia rating (CDR) score of 0 (56 subjects) or 0.5 (16 subjects). Subject race/ethnicities were self-reported as: White (53), Asian (12), American Indian or Alaska Native (3), more than one race (4), race not reported (1); Hispanic/Latino (any race) (10), non-Hispanic/Latino (any race) 62. MR images were acquired on a 3 Tesla Siemens Prisma scanner at the University of Southern California Alzheimer's Disease Research Center (ADRC). Anatomical sagittal T1-weighted magnetization prepared rapid acquisition gradient-echo (MPRAGE) scan parameters were acquired (TR 2300 ms; TE 2.95 ms; 1.2×1.0×1.0 mm³ voxel size). The inventors also acquired a 64-direction (b=1000 s/mm2) diffusion MRI scan (TR 7100 ms; TE 71.0 ms; 2.5×2.5×2.5 mm³ voxel size). All scans were visually assessed for quality. For each subject, the diffusion images were denoised with MATLAB version R2014b software (MathWorks, Natick, MA) using a local primary components analysis (LPCA) tool with the Rician filter, with intensity bias correction. Distortion correction of DWI included correction for Gibbs ringing using MRtrix3 and eddy current correction using the eddy_correct tool in FSL utilities (FSL 5.0.9; (fmrib.ox.ac.uk/fsl). The inventors performed bias field correction using MRtrix3. Echo planar imaging (EPI) susceptibility artifacts were corrected using FSL and ANTS software to align the average b0 maps to subject-specific T1-weighted MPRAGE structural scan. Each step was visually quality checked. Fractional anisotropy (FA) maps—indicating diffusion restriction within a voxel—were created using FSL software. FA is a metric of microstructural integrity shown to augment the power to detect AD-specific deficits with lower FA values typically representing poor white matter microstructural integrity in AD. Voxelwise statistical analysis of DWI FA data was performed using the FSL-based tool, tract based-spatial statistics (TBSS). TBSS applies nonlinear registration to bring all FA maps into standard template space. A mean FA skeleton was created and then thresholded at 0.2, resulting in a 4D skeletonised FA image used in voxelwise statistical analyses detailed below. We used the general linear model (GLM) with FSL's Threshold-Free Cluster Enhancement (TFCE) option to evaluate the relationship between SHMOOSE and voxelwise white-matter FA within the mean FA skeleton, covarying for CDR score, age, and reported sex. SHMOOSE values<100 were coded as 50 for this analysis (5 subjects). For CSF tau and p tau 181, linear regression analysis was conducted with biological sex and age as a covariate, and SHMOOSE CSF levels as the dependent variable. Separately, we considered the effects for p tau 181 were mediated through total tau levels. Hence, we used the mediation package in R and modeled the effect of p tau 181, age, and biological sex on the mediator (i.e., tau); modeled the effect of tau, p tau 181, biological sex, and age on SHMOOSE; and used these models to determine the indirect (ACME) and direct (ADE) effects using the mediate function.

Human Brain SHMOOSE mtSNP Differential Expression Analysis

The inventors utilized genotype and transcriptome data generated by Mayo (Synapse ID: syn5550404). “RNAseq TCX” data were analyzed by SHMOOSE genotype. Subject SHMOOSE genotype was extracted from Mayo LOAD GWAS data that was generated from the HumanHap300-Duo Genotyping BeadChips. A complete description of the processing and individual sub cohorts has been described previously. Briefly, gene expression fastq files from human brain temporal cortex were aligned using the Mayo MAP-Rseq pipeline. Normalized read counts were then examined for differential expression by SHMOOSE genotype using multi-variable linear regression to adjust for age at death, biological sex, and RNA integrity. Source code in R provided by Mayo was modified to conduct the differential expression analysis by SHMOOSE genotype. Results contain all genes that have non-zero raw counts in at least 1 subject, and each gene contains a beta value representing the effect size by SHMOOSE mtSNP. Multiple hypothesis correction was performed using Benjamin Hochberg. Significant genes were included in gene enrichment analyses using the clusterProfiler package in R. By using the enrichGo and enrichWP functions, significantly enriched pathways were extracted according to a hypergeometric model. A total of 14 SHMOOSE mtSNP carriers were assessed against 55 reference allele SHMOOSE individuals. These samples were selected by extracting non-demented individuals at time of death that also contained SHMOOSE genotype data.

### SHMOOSE-Treated Cell Transcriptomics

Differentiated neural cells were incubated with 10 uM SHMOOSE or SHMOOSE.D47N for 24 hours followed by rapid RNA extraction. Cells were washed once with ice-cold DPBS and immediately lysed with TRIzol (Thermo Scientific), and RNA was extracted using the Quick-RNA Miniprep Kit (Zymo Research). High quality RNA used for library preparation (mRNA-Seq Nu Quant), which captures poly-adenylated RNA. From there, prepped samples were sequenced on an Illumina NextSeq 550 platform for 75 single end cycles. Each sample achieved a read depth of nearly 25 million. High quality fastq files were ensured using FastQC and mapped to the human reference genome (GRCh38.p13) using kallisto. Normalized fold changes were then used to estimate differential gene expression between SHMOOSE and SHMOOSE.D47N treated cells using the DESeq2 package in R. Gene enrichment was carried out on significantly different gene (FDR<0.2) using the clusterProfiler package in R.

### SHMOOSE In Vivo

To examine the transcriptomes of mice treated with SHMOOSE, 12-week-old male C57Bl/6N mice were obtained from The Jackson Laboratory. Mice were fed a high fat diet for 10 days (60% total calories) prior to initiation of SHMOOSE daily IP injections (2.5 mg/kg). No more than 60 ul of volume were injected IP. After two weeks of IP injections, mice were euthanized following food withdrawal overnight, then brain was rapidly removed, hypothalamus extracted, and hemisected midsagittal. Hemibrains were further microdissected to extract the hippocampus and cortex. Tissues were snap frozen and RNA was extracted by adding 100 ul of TRIzol (Thermo Scientific) per 10 mg tissue. Homogenates were then spun down at 16,000 RCF for 60 seconds and processed using the Quick-RNA Miniprep Kit (Zymo Research). High quality RNA used for library preparation (mRNA-Seq Nu Quant), which captures poly-adenylated RNA. From there, prepped samples were sequenced on an Illumina NextSeq 550 platform for 75 single end cycles and fastq files were quality ensured using FastQC and mapped to the mouse reference genome (GRCm39) using kallisto. Normalized fold changes were then used to estimate differential gene expression for SHMOOSE-treated mice using the DESeq2 package in R. Gene enrichment was carried out on significantly different gene (FDR<0.2) using the clusterProfiler package in R.

### Seahorse Assay

SH-SY5Y Cells were Plated into 96-Well Plates at a Density of 10,000 Cells. The following day, cells were differentiated for a total of 4 days. Thereafter, SHMOOSE or SHMOOSE.D47N were incubated for 24 hours, followed by cell real-time oxygen consumption rates measurements using XF96 Extracellular Flux Analyzer (Seahorse Bioscience). ATP turnover and maximum respiratory capacity were calculated after challenging cells with oligomycin and FCCP (carbonyl cyanide 4-[trifluoromethoxy]phenylhydrazone). Additionally, glycolytic rate was determined using extracellular acidification rate (ECAR) and individually reported relative to basal level in percentage. All readings were normalized to total DNA content using Hoechst 33342.

SHMOOSE Differential Expression in iPSCs and AD Brains

RNA-Seq data from neurons derived from iPSCs with 694 FAD mutations were downloaded to test SHMOOSE expression as a function of FAD mutations (GEO: GSE128343). Fastq files were aligned to the human reference genome (GRCh38.p13) using STAR with default parameters. Aligned BAM files were loaded into R using the BioConductor package. A custom GTF file containing the SHMOOSE genomic coordinates and other mitochondrial genes were used for the differential expression analysis. Counts were normalized to mitochondrial read count. Counts were called using the “union” mode by the summarizeOverlaps function. Differential expression analysis was conducted using negative binomial regression by the DESeq2 package in R. The inventors also used Mayo RNASeq data (Synapse ID: syn5550404) to assess SHMOOSE RNA differences by AD and by genotype, following the same processing workflow.

### Amyloid Beta Toxicity Assay

SH-SY5Y cells were differentiated for 4 days, incubated with 10 uM SHMOOSE or SHMOOSE.D47N for 24 hours followed by another incubation with SHMOOSE or SHMOOSE.D47N with or without oligomerized 1 uM amyloid beta 42 (CPC Scientific), prepared as formerly described. A two-color fluorescence cell viability assay (LIVE/DEAD Viability/Cytotoxicity Kit; Invitrogen (cat. L3224) was used to distinguish live cells from dead cells after humanin and amyloid beta 42 treatment. The ratio of live to dead cells can be quantified since live cells retain the Calcein AM dye and dead cells with damaged membranes permit entry of the ethidium homodimer dye.

### SHMOOSE-Mitofilin Protein Interaction

To identify the SHMOOSE interactome, multiple experiments were conducted. First, 1.5 nmol of SHMOOSE was spiked into 1 mg SH-SY5Y cell lysate for 6 hours at 4° C. Lysates were prepared using Thermo Pierce CoIP lysis buffer with 1× Thermo protease inhibitor cocktail and 1 mM PMSF. Briefly, cells were washed with ice-cold DPBS twice, lysed for 15 minutes on ice, and centrifuged for 10 minutes at 12,000 RCF at 4° C. The rationale for conducting this experiment was to ensure identical protein amounts between conditions and avoid differential protein expression caused by SHMOOSE treatment to cells. After 6 hours, SHMOOSE was immunoprecipitated from samples using Dynabeads A conjugated to 5 ug of custom c-terminus SHMOOSE antibody. As a negative control, SHMOOSE-spiked lysates were also immunoprecipitated using 5 ug rabbit IgG. Proteins were eluted from beads using 50 mM Glycine pH 2.8, and eluents were pH neutralized using Tris HCl pH 7.5. Complete eluents were then processed for protein identification using LC-MS. Samples were mixed with same volume of digestion buffer (8M Urea, 0.1M Tris-HCl pH 8.5), then each sample was reduced and alkylated via sequential 20-minute incubations with 5 mM TCEP and 10 mM iodoacetamide at room temperature in the dark while being mixed at 1200 rpm in an Eppendorf thermomixer. 6 μl of carboxylate-modified magnetic beads (CMMB and widely known as SP3) was added to each sample. Ethanol was added to a concentration of 50% to induce protein binding to CMMB. CMMB were washed 3 times with 80% ethanol and then resuspended with 50 μl 50 mM TEAB. The protein was digested overnight with 0.1 μg LysC (Promega) and 0.8 μg trypsin (Pierce) at 37° C. Following digestion, 1 ml of 100% acetonitrile was added to each to sample to increase the final acetonitrile concentration to over 95% to induce peptide binding to CMMB. CMMB were then washed 3 times with 100% acetonitrile and the peptide was eluted with 50 μl of 2% DMSO. Eluted peptide samples were dried by vacuum centrifugation and reconstituted in 5% formic acid before analysis by LC-MS/MS. Peptide samples were separated on a 75 μM ID, 25 cm C18 column packed with 1.9 μM C18 particles (Dr. Maisch GmbH HPLC) using a 140-minute gradient of increasing acetonitrile concentration and injected into a Thermo Orbitrap-Fusion Lumos Tribrid mass spectrometer. MS/MS spectra were acquired using Data Dependent Acquisition (DDA) mode. MS/MS database searching was performed using MaxQuant (1.6.10.43) against the human reference proteome from EMBL (UP000005640_9606 HUMAN Homo sapiens, 20874 entries). Statistical analysis of MaxQuant label-free quantitation data was performed with the artMS Bioconductor package, which performs the relative quantification of protein abundance using the MSstats Bioconductor package (default parameters). The abundance of proteins missing from one condition but found in more than 2 biological replicates of the other condition for any given comparison were estimated by imputing intensity values from the lowest observed MS1-intensity across samples and p values were randomly assigned to those between 0.05 and 0.01 for illustration purposes. The inventors chose to target mitofilin (IMMT) based on imputed fold change and p value thresholds.

Second, the inventors validated the mitofilin interaction identified from MS using a series of reciprocal co-immunoprecipitation experiments with SHMOOSE antibody and mitofilin antibody. The inventors treated differentiated SH-SY5Y cells for 30 minutes with 1 uM SHMOOSE and lysed cells using Thermo Pierce CoIP lysis buffer as mentioned above. Thereafter, the inventors incubated samples with 5 ug of SHMOOSE antibody, mitofilin antibody, or negative rabbit IgG for 30 minutes at room temperature. Antibody-coupled Dynabeads A were washed 3 times with TBST 0.1% and eluted using Glycine pH 2.8, NuPAGE LDS sample buffer, and NuPAGE sample reducing agent for 5 minutes at 95 C. Eluents were then loaded into NuPAGE 4-12% Bis-Tris gels for electrophoresis. Migrated proteins were transferred to PVDF membranes, blocked with 5% BSA in TBS 0.1% tween, and incubated with respective antibodies at 1:1000 dilutions overnight at 4° C. The next day, membranes were washed with TBST 0.1% and incubated with 1:30,000 secondary antibody conjugated to HRP against the respective primary antibody species of origin, then excited using ECl reagent for 5 minutes.

Third, the inventors conducted reciprocal dot blots for SHMOOSE, SHMOOSE.D47N, and mitofilin by immobilizing 140 ng of recombinant mitofilin (OriGene), SHMOOSE, or SHMOOSE.D47N on nitrocellulose membranes. After proteins were dried, membranes were blocked for 30 minutes with SuperBlock (PBS) blocking buffer (Thermo) at room temperature. Then, either SHMMOOSE or mitofilin were flowed over blocked membranes at a concentration at 1 ug/ml for 30 minutes at room temperature in blocking buffer. Membranes were washed three times for five minutes each with TBSBT 0.1% and then incubated with 0.5 ug/ml of respective antibodies for 30 minutes at room temperature in blocking buffer. Membranes were washed three times for five minutes each with TSBT 0.1% and incubated with 1:30,000 secondary antibodies against species of primary antibody origin. Membranes were washed three times for five minutes each with TBST 0.1%, followed by excitation using ECl reagent for 1 minute.

### MTT Assay

MTT assays were used to measure the effect of mitofilin knockdown. SH-SY5Y cells were reverse transfected using RNAiMAX (Invitrogen) and 40 nM mitofilin siRNA (Horizon, SMARTpool) when plated into 96-well plates at a density 784 of 10,000 cells. The following day, cells were differentiated for a total of 4 days and transfected with another 40 nM mitofilin siRNA. Two days later, differentiation medium was changed with an addition 40 nM mitofilin siRNA. 24 hours before the MTT assay, cells were treated with 10 uM SHMOOSE or solvent control. MTT (Sigma-Aldrich) reagent (5 mg/ml) was added to each well after treatments for four hours and lysed before absorbance values were read using the SpectrMax M3 microplate reader.

### SHMOOSE-Mitofilin Docking Model

To model the IMMT-SHMOOSE interaction, the inventors used HDOCK, which is a hybrid algorithm of template-based modeling and ab initio free docking, as described elsewhere. Mitofilin was considering the “receptor” and SHMOOSE was considered the “ligand.”

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the compositions and methods related to induced mitochondrial peptides, analogues and derivatives thereof, methods and compositions related to use of the aforementioned compositions, techniques and composition and use of solutions used therein, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A composition comprising:
a mitochondrial peptide having an amino acid sequence MPPCLTTWLSQLLKDNSYPLVLGPKNFGATPNKSNNHAHYYNHPNPDFPNSPHP YHPR (SEQ ID NO: 93), or a fragment, an analog, or a derivative thereof.

2. The composition of claim 1, wherein the mitochondrial peptide comprises the amino acid sequence
(SEQ ID NO: 93)
MPPCLTTWLSQLLKDNSYPLVLGPKNFGATPNKSNNHAHYYNHPNPDFP
NSPHPYHPR.

3. The composition of claim 1, wherein the mitochondrial peptide comprises an amino acid sequence of any of one of SEQ ID NO:1-SEQ ID NO:92, an amino acid sequence of any of one of SEQ ID NO:97-SEQ ID NO: 107, or an amino acid sequence of PCLTTWLSQLLKDNSYPLVLGPKNF (SEQ ID NO: 3).

4. (canceled)

5. The composition of claim 1, wherein the mitochondrial peptide comprises an amino acid sequence with about 20%, 25%, 30%, 35%, 40%, 45%, 50%, 55%, 60%, 65%, 70%, 75%, 80%, 85%, 90%, 95%, or more percentage identity to
(SEQ ID NO: 93)
MPPCLTTWLSQLLKDNSYPLVLGPKNFGATPNKSNNHAHYYNHPNPDFP
NSPHPYHPR,
or to
(SEQ ID NO: 3)
PCLTTWLSQLLKDNSYPLVLGPKNF
and/or wherein the mitochondrial peptide is 19-70 amino acids in length.

6. (canceled)

7. The composition of claim 1, wherein the mitochondrial peptide possesses a post-translational or artificial modification, the artificial modification comprising pegylation, fatty-acid conjugation, polypeptide extension, IgG-Fc, CPT, HSA, ELP, transferrin, or albumin modification.

8. (canceled)

9. The composition of claim 1, further comprising a pharmaceutically acceptable excipient or pharmaceutically acceptable carrier.

10. A method of treating a disease and/or condition comprising:
administering a quantity of the composition of claim 1 to a subject in need of treatment of the disease and/or condition.

11. The method of claim 10, wherein the mitochondrial peptide is a fragment of the amino acid sequence of SEQ ID NO: 93, wherein the fragment comprises the amino acid sequence PCLTTWLSQLLKDNSYPLVLGPKNF (SEQ ID NO: 3), and/or the mitochondrial peptide is 19-70 amino acids in length.

12. (canceled)

13. The method of claim 10, wherein the disease and/or condition comprises a neurodegenerative disease and/or condition, optionally Alzheimer's disease or characterized by a level of amyloid beta above a reference.

14. The method of claim 13, wherein the mitochondrial peptide increases tau levels in cerebrospinal fluid of the subject, wherein the mitochondrial peptide decreases amyloid beta levels or amyloid beta plaques in a brain of the subject, and/or wherein the mitochondrial peptide reduces or inhibits a decrease in volume of interior temporal cortex tissue of the subject.

15. (canceled)

16. (canceled)

17. The method of claim 13, wherein the subject is a carrier of single nucleotide polymorphism (SNP) rs2853499 having an “A” allele at the SNP position.

18. The method of claim 13, wherein the neurodegenerative disease and/or condition is Parkinson's disease.

19. The method of claim 18, wherein the mitochondrial peptide reduces or inhibits decrease in volume of superior parietal lobe cortex tissue of the subject.

20. The method of claim 10, wherein the subject expresses high amounts of MPPCLTTWLSQLLKDNSYPLVLGPKNFGATPNKSNNHAHYYNHPNPDFPNSPHP YHPR (SEQ ID NO: 93) measured in a biological sample relative to a healthy normal subject.

21. A method of detecting one or more biomarkers, comprising:
detecting the presence, absence, or expression level of one or more biomarkers in a biological sample obtained from a subject desiring a determination regarding the one or more biomarkers; and
detecting the presence, absence, or expression level of the one or more biomarkers,
wherein the one or more biomarkers comprises a peptide of the sequence MPPCLTTWLSQLLKDNSYPLVLGPKNFGATPNKSNNHAHYYNHPNPDFPNSPHP YHPR (SEQ ID NO: 93), MPPCLTTWLSQLLKDNSYPLVLGPKNFGATPNKSNNHAHYYNHPNPNFPNSPHP YHPR (SEQ ID NO: 94), or a single nucleotide polymorphism (SNP) rs2853499.

22. The method of claim 21, wherein detecting the presence, absence, or expression level comprises an immunoassay.

23. The method of claim 21, wherein the one or more biomarkers comprises a single nucleotide polymorphism (SNP) rs2853499, wherein an “A” allele is at the SNP position.

24. The method of claim 21, further comprising:
diagnosing the subject with a disease and/or condition or with an increased likelihood of having the disease and/or condition when the presence of the peptide having SEQ ID NO:94 is detected, OR
diagnosing the subject with a disease and/or condition or with an increased likelihood of having the disease and/or condition when low expression levels of the peptide having SEQ ID NO:93, as compared to a healthy control, is detected, OR
diagnosing the subject with a disease and/or condition when a single nucleotide polymorphism (SNP) rs2853499, wherein an “A” allele is at the SNP position, is detected,
wherein the disease and/or condition is a neurodegenerative disease and/or condition.

25. The method of claim 24, wherein the neurodegenerative disease and/or condition is selected from the group consisting of Alzheimer's disease, Parkinson's disease, dementia, and combinations thereof.

26. A method of detecting a genotype of a mitochondrial-derived peptide in a subject in need thereof, comprising:
assaying a biological sample obtained from the subject to detect genotype at a single nucleotide polymorphism (SNP), wherein the SNP is rs2853499.

27. The method of claim 26, further comprising detecting an A allele at the SNP in the subject and/or wherein the detection detects a count of the A allele at the SNP in the subject higher than that in a control subject, wherein the subject has Alzheimer's disease or has a risk factor for developing Alzheimer's disease.

28. (canceled)

29. The method of claim 27, further comprising administering an Alzheimer's disease therapy to the subject and/or wherein the subject desires a determination regarding a neurodegeneration disease or disorder, optionally Alzheimer's disease.

30. (canceled)


SEQUENCE LISTING (112 sequences)

SEQ ID NO: 1 - protein, 25 aa, Artificial Sequence
  synthetic construct
MPPCLTTWLSQLLKDNSYPLVLGPK

SEQ ID NO: 2 - protein, 25 aa, Artificial Sequence
  synthetic construct
PPCLTTWLSQLLKDNSYPLVLGPKN

SEQ ID NO: 3 - protein, 25 aa, Artificial Sequence
  synthetic construct
PCLTTWLSQLLKDNSYPLVLGPKNF

SEQ ID NO: 4 - protein, 25 aa, Artificial Sequence
  synthetic construct
CLTTWLSQLLKDNSYPLVLGPKNFG

SEQ ID NO: 5 - protein, 25 aa, Artificial Sequence
  synthetic construct
LTTWLSQLLKDNSYPLVLGPKNFGA

SEQ ID NO: 6 - protein, 25 aa, Artificial Sequence
  synthetic construct
TTWLSQLLKDNSYPLVLGPKNFGAT

SEQ ID NO: 7 - protein, 25 aa, Artificial Sequence
  synthetic construct
TWLSQLLKDNSYPLVLGPKNFGATP

SEQ ID NO: 8 - protein, 25 aa, Artificial Sequence
  synthetic construct
WLSQLLKDNSYPLVLGPKNFGATPN

SEQ ID NO: 9 - protein, 25 aa, Artificial Sequence
  synthetic construct
LSQLLKDNSYPLVLGPKNFGATPNK

SEQ ID NO: 10 - protein, 25 aa, Artificial Sequence
  synthetic construct
SQLLKDNSYPLVLGPKNFGATPNKS

SEQ ID NO: 11 - protein, 25 aa, Artificial Sequence
  synthetic construct
QLLKDNSYPLVLGPKNFGATPNKSN

SEQ ID NO: 12 - protein, 25 aa, Artificial Sequence
  synthetic construct
LLKDNSYPLVLGPKNFGATPNKSNN

SEQ ID NO: 13 - protein, 25 aa, Artificial Sequence
  synthetic construct
LKDNSYPLVLGPKNFGATPNKSNNH

SEQ ID NO: 14 - protein, 25 aa, Artificial Sequence
  synthetic construct
KDNSYPLVLGPKNFGATPNKSNNHA

SEQ ID NO: 15 - protein, 25 aa, Artificial Sequence
  synthetic construct
DNSYPLVLGPKNFGATPNKSNNHAH

SEQ ID NO: 16 - protein, 25 aa, Artificial Sequence
  synthetic construct
NSYPLVLGPKNFGATPNKSNNHAHY

SEQ ID NO: 17 - protein, 25 aa, Artificial Sequence
  synthetic construct
SYPLVLGPKNFGATPNKSNNHAHYY

SEQ ID NO: 18 - protein, 25 aa, Artificial Sequence
  synthetic construct
YPLVLGPKNFGATPNKSNNHAHYYN

SEQ ID NO: 19 - protein, 25 aa, Artificial Sequence
  synthetic construct
PLVLGPKNFGATPNKSNNHAHYYNH

SEQ ID NO: 20 - protein, 25 aa, Artificial Sequence
  synthetic construct
LVLGPKNFGATPNKSNNHAHYYNHP

SEQ ID NO: 21 - protein, 25 aa, Artificial Sequence
  synthetic construct
VLGPKNFGATPNKSNNHAHYYNHPN

SEQ ID NO: 22 - protein, 25 aa, Artificial Sequence
  synthetic construct
LGPKNFGATPNKSNNHAHYYNHPNP

SEQ ID NO: 23 - protein, 25 aa, Artificial Sequence
  synthetic construct
GPKNFGATPNKSNNHAHYYNHPNPD

SEQ ID NO: 24 - protein, 25 aa, Artificial Sequence
  synthetic construct
PKNFGATPNKSNNHAHYYNHPNPDF

SEQ ID NO: 25 - protein, 25 aa, Artificial Sequence
  synthetic construct
KNFGATPNKSNNHAHYYNHPNPDFP

SEQ ID NO: 26 - protein, 25 aa, Artificial Sequence
  synthetic construct
NFGATPNKSNNHAHYYNHPNPDFPN

SEQ ID NO: 27 - protein, 25 aa, Artificial Sequence
  synthetic construct
FGATPNKSNNHAHYYNHPNPDFPNS

SEQ ID NO: 28 - protein, 25 aa, Artificial Sequence
  synthetic construct
GATPNKSNNHAHYYNHPNPDFPNSP

SEQ ID NO: 29 - protein, 25 aa, Artificial Sequence
  synthetic construct
ATPNKSNNHAHYYNHPNPDFPNSPH

SEQ ID NO: 30 - protein, 25 aa, Artificial Sequence
  synthetic construct
TPNKSNNHAHYYNHPNPDFPNSPHP

SEQ ID NO: 31 - protein, 25 aa, Artificial Sequence
  synthetic construct
PNKSNNHAHYYNHPNPDFPNSPHPY

SEQ ID NO: 32 - protein, 25 aa, Artificial Sequence
  synthetic construct
NKSNNHAHYYNHPNPDFPNSPHPYH

SEQ ID NO: 33 - protein, 25 aa, Artificial Sequence
  synthetic construct
KSNNHAHYYNHPNPDFPNSPHPYHP

SEQ ID NO: 34 - protein, 25 aa, Artificial Sequence
  synthetic construct
SNNHAHYYNHPNPDFPNSPHPYHPR

SEQ ID NO: 35 - protein, 25 aa, Artificial Sequence
  synthetic construct
GPKNFGATPNKSNNHAHYYNHPNPN

SEQ ID NO: 36 - protein, 25 aa, Artificial Sequence
  synthetic construct
PKNFGATPNKSNNHAHYYNHPNPNF

SEQ ID NO: 37 - protein, 25 aa, Artificial Sequence
  synthetic construct
KNFGATPNKSNNHAHYYNHPNPNFP

SEQ ID NO: 38 - protein, 25 aa, Artificial Sequence
  synthetic construct
NFGATPNKSNNHAHYYNHPNPNFPN

SEQ ID NO: 39 - protein, 25 aa, Artificial Sequence
  synthetic construct
FGATPNKSNNHAHYYNHPNPNFPNS

SEQ ID NO: 40 - protein, 25 aa, Artificial Sequence
  synthetic construct
GATPNKSNNHAHYYNHPNPNFPNSP

SEQ ID NO: 41 - protein, 25 aa, Artificial Sequence
  synthetic construct
ATPNKSNNHAHYYNHPNPNFPNSPH

SEQ ID NO: 42 - protein, 25 aa, Artificial Sequence
  synthetic construct
TPNKSNNHAHYYNHPNPNFPNSPHP

SEQ ID NO: 43 - protein, 25 aa, Artificial Sequence
  synthetic construct
PNKSNNHAHYYNHPNPNFPNSPHPY

SEQ ID NO: 44 - protein, 25 aa, Artificial Sequence
  synthetic construct
NKSNNHAHYYNHPNPNFPNSPHPYH

SEQ ID NO: 45 - protein, 25 aa, Artificial Sequence
  synthetic construct
KSNNHAHYYNHPNPNFPNSPHPYHP

SEQ ID NO: 46 - protein, 25 aa, Artificial Sequence
  synthetic construct
SNNHAHYYNHPNPNFPNSPHPYHPR

SEQ ID NO: 47 - protein, 25 aa, Artificial Sequence
  synthetic construct
MPPCLDDWLDQLLKDNDDPLVLGPK

SEQ ID NO: 48 - protein, 25 aa, Artificial Sequence
  synthetic construct
PPCLDDWLDQLLKDNDDPLVLGPKN

SEQ ID NO: 49 - protein, 25 aa, Artificial Sequence
  synthetic construct
PCLDDWLDQLLKDNDDPLVLGPKNF

SEQ ID NO: 50 - protein, 25 aa, Artificial Sequence
  synthetic construct
CLDDWLDQLLKDNDDPLVLGPKNFG

SEQ ID NO: 51 - protein, 25 aa, Artificial Sequence
  synthetic construct
LDDWLDQLLKDNDDPLVLGPKNFGA

SEQ ID NO: 52 - protein, 25 aa, Artificial Sequence
  synthetic construct
DDWLDQLLKDNDDPLVLGPKNFGAD

SEQ ID NO: 53 - protein, 25 aa, Artificial Sequence
  synthetic construct
DWLDQLLKDNDDPLVLGPKNFGADP

SEQ ID NO: 54 - protein, 25 aa, Artificial Sequence
  synthetic construct
WLDQLLKDNDDPLVLGPKNFGADPN

SEQ ID NO: 55 - protein, 25 aa, Artificial Sequence
  synthetic construct
LDQLLKDNDDPLVLGPKNFGADPNK

SEQ ID NO: 56 - protein, 25 aa, Artificial Sequence
  synthetic construct
DQLLKDNDDPLVLGPKNFGADPNKD

SEQ ID NO: 57 - protein, 25 aa, Artificial Sequence
  synthetic construct
QLLKDNDDPLVLGPKNFGADPNKDN

SEQ ID NO: 58 - protein, 25 aa, Artificial Sequence
  synthetic construct
LLKDNDDPLVLGPKNFGADPNKDNN

SEQ ID NO: 59 - protein, 25 aa, Artificial Sequence
  synthetic construct
LKDNDDPLVLGPKNFGADPNKDNNH

SEQ ID NO: 60 - protein, 25 aa, Artificial Sequence
  synthetic construct
KDNDDPLVLGPKNFGADPNKDNNHA

SEQ ID NO: 61 - protein, 25 aa, Artificial Sequence
  synthetic construct
DNDDPLVLGPKNFGADPNKDNNHAH

SEQ ID NO: 62 - protein, 25 aa, Artificial Sequence
  synthetic construct
NDDPLVLGPKNFGADPNKDNNHAHD

SEQ ID NO: 63 - protein, 25 aa, Artificial Sequence
  synthetic construct
DDPLVLGPKNFGADPNKDNNHAHDD

SEQ ID NO: 64 - protein, 25 aa, Artificial Sequence
  synthetic construct
DPLVLGPKNFGADPNKDNNHAHDDN

SEQ ID NO: 65 - protein, 25 aa, Artificial Sequence
  synthetic construct
PLVLGPKNFGADPNKDNNHAHDDNH

SEQ ID NO: 66 - protein, 25 aa, Artificial Sequence
  synthetic construct
LVLGPKNFGADPNKDNNHAHDDNHP

SEQ ID NO: 67 - protein, 25 aa, Artificial Sequence
  synthetic construct
VLGPKNFGADPNKDNNHAHDDNHPN

SEQ ID NO: 68 - protein, 25 aa, Artificial Sequence
  synthetic construct
LGPKNFGADPNKDNNHAHDDNHPNP

SEQ ID NO: 69 - protein, 25 aa, Artificial Sequence
  synthetic construct
GPKNFGADPNKDNNHAHDDNHPNPD

SEQ ID NO: 70 - protein, 25 aa, Artificial Sequence
  synthetic construct
PKNFGADPNKDNNHAHDDNHPNPDF

SEQ ID NO: 71 - protein, 25 aa, Artificial Sequence
  synthetic construct
KNFGADPNKDNNHAHDDNHPNPDFP

SEQ ID NO: 72 - protein, 25 aa, Artificial Sequence
  synthetic construct
NFGADPNKDNNHAHDDNHPNPDFPN

SEQ ID NO: 73 - protein, 25 aa, Artificial Sequence
  synthetic construct
FGADPNKDNNHAHDDNHPNPDFPND

SEQ ID NO: 74 - protein, 25 aa, Artificial Sequence
  synthetic construct
GADPNKDNNHAHDDNHPNPDFPNDP

SEQ ID NO: 75 - protein, 25 aa, Artificial Sequence
  synthetic construct
ADPNKDNNHAHDDNHPNPDFPNDPH

SEQ ID NO: 76 - protein, 25 aa, Artificial Sequence
  synthetic construct
DPNKDNNHAHDDNHPNPDFPNDPHP

SEQ ID NO: 77 - protein, 25 aa, Artificial Sequence
  synthetic construct
PNKDNNHAHDDNHPNPDFPNDPHPD

SEQ ID NO: 78 - protein, 25 aa, Artificial Sequence
  synthetic construct
NKDNNHAHDDNHPNPDFPNDPHPDH

SEQ ID NO: 79 - protein, 25 aa, Artificial Sequence
  synthetic construct
KDNNHAHDDNHPNPDFPNDPHPDHP

SEQ ID NO: 80 - protein, 25 aa, Artificial Sequence
  synthetic construct
DNNHAHDDNHPNPDFPNDPHPDHPR

SEQ ID NO: 81 - protein, 25 aa, Artificial Sequence
  synthetic construct
GPKNFGADPNKDNNHAHDDNHPNPN

SEQ ID NO: 82 - protein, 25 aa, Artificial Sequence
  synthetic construct
PKNFGADPNKDNNHAHDDNHPNPNF

SEQ ID NO: 83 - protein, 25 aa, Artificial Sequence
  synthetic construct
KNFGADPNKDNNHAHDDNHPNPNFP

SEQ ID NO: 84 - protein, 25 aa, Artificial Sequence
  synthetic construct
NFGADPNKDNNHAHDDNHPNPNFPN

SEQ ID NO: 85 - protein, 25 aa, Artificial Sequence
  synthetic construct
FGADPNKDNNHAHDDNHPNPNFPND

SEQ ID NO: 86 - protein, 25 aa, Artificial Sequence
  synthetic construct
GADPNKDNNHAHDDNHPNPNFPNDP

SEQ ID NO: 87 - protein, 25 aa, Artificial Sequence
  synthetic construct
ADPNKDNNHAHDDNHPNPNFPNDPH

SEQ ID NO: 88 - protein, 25 aa, Artificial Sequence
  synthetic construct
DPNKDNNHAHDDNHPNPNFPNDPHP

SEQ ID NO: 89 - protein, 25 aa, Artificial Sequence
  synthetic construct
PNKDNNHAHDDNHPNPNFPNDPHPD

SEQ ID NO: 90 - protein, 25 aa, Artificial Sequence
  synthetic construct
NKDNNHAHDDNHPNPNFPNDPHPDH

SEQ ID NO: 91 - protein, 25 aa, Artificial Sequence
  synthetic construct
KDNNHAHDDNHPNPNFPNDPHPDHP

SEQ ID NO: 92 - protein, 25 aa, Artificial Sequence
  synthetic construct
DNNHAHDDNHPNPNFPNDPHPDHPR

SEQ ID NO: 93 - protein, 58 aa, Homo sapiens
MPPCLTTWLSQLLKDNSYPLVLGPKNFGATPNKSNNHAHYYNHPNPDFPNSPHPYHPR

SEQ ID NO: 94 - protein, 58 aa, Artificial Sequence
  synthetic construct
MPPCLTTWLSQLLKDNSYPLVLGPKNFGATPNKSNNHAHYYNHPNPNFPNSPHPYHPR

SEQ ID NO: 95 - protein, 14 aa, Artificial Sequence
  synthetic construct
PCLTTWLSQLLKDN

SEQ ID NO: 96 - protein, 11 aa, Artificial Sequence
  synthetic construct
DNSYPLVLGPK

SEQ ID NO: 97 - protein, 58 aa, Artificial Sequence
  synthetic construct
MPPCITTWLSQLLKDNSYPLVLGPKNFGATPNKSNNHAHYYNHPNPDFPNSPHPYHPR

SEQ ID NO: 98 - protein, 58 aa, Artificial Sequence
  synthetic construct
MPPCITTWISQLLKDNSYPLVLGPKNFGATPNKSNNHAHYYNHPNPDFPNSPHPYHPR

SEQ ID NO: 99 - protein, 58 aa, Artificial Sequence
  synthetic construct
MPPCITTWISQILKDNSYPLVLGPKNFGATPNKSNNHAHYYNHPNPDFPNSPHPYHPR

SEQ ID NO: 100 - protein, 58 aa, Artificial Sequence
  synthetic construct
MPPCITTWISQIIKDNSYPLVLGPKNFGATPNKSNNHAHYYNHPNPDFPNSPHPYHPR

SEQ ID NO: 101 - protein, 58 aa, Artificial Sequence
  synthetic construct
MPPCLTTWISQIIKDNSYPLVLGPKNFGATPNKSNNHAHYYNHPNPDFPNSPHPYHPR

SEQ ID NO: 102 - protein, 58 aa, Artificial Sequence
  synthetic construct
MPPCLTTWLSQIIKDNSYPLVLGPKNFGATPNKSNNHAHYYNHPNPDFPNSPHPYHPR

SEQ ID NO: 103 - protein, 58 aa, Artificial Sequence
  synthetic construct
MPPCLTTWLSQLIKDNSYPLVLGPKNFGATPNKSNNHAHYYNHPNPDFPNSPHPYHPR

SEQ ID NO: 104 - protein, 58 aa, Artificial Sequence
  synthetic construct
MPSCLTTWLSQLLKDNSYPLVLGPKNFGATPNKSNNHAHYYNHPNPDFPNSPHPYHPR

SEQ ID NO: 105 - protein, 58 aa, Artificial Sequence
  synthetic construct
MPSCLTTWLSSLLKDNSYPLVLGPKNFGATPNKSNNHAHYYNHPNPDFPNSPHPYHPR

SEQ ID NO: 106 - protein, 58 aa, Artificial Sequence
  synthetic construct
MPSCLTTWLSSLLSDNSYPLVLGPKNFGATPNKSNNHAHYYNHPNPDFPNSPHPYHPR

SEQ ID NO: 107 - protein, 58 aa, Artificial Sequence
  synthetic construct
MPSCLTTWISSIISDNSYPLVLGPKNFGATPNKSNNHAHYYNHPNPDFPNSPHPYHPR

SEQ ID NO: 108 - protein, 11 aa, Artificial Sequence
  synthetic construct
PCLTTWLSQLL

SEQ ID NO: 109 - protein, 11 aa, Artificial Sequence
  synthetic construct
CLTTWLSQLLK

SEQ ID NO: 110 - protein, 11 aa, Artificial Sequence
  synthetic construct
LTTWLSQLLKD

SEQ ID NO: 111 - protein, 6 aa, Artificial Sequence
  synthetic construct
CWLLLL

SEQ ID NO: 112 - protein, 5 aa, Artificial Sequence
  synthetic construct
WLLLL